(19) United States
(12) Patent Application Publication
(10) Pub. No.: US 2019/0201557 A1
(43) Pub. Date: Jul. 4, 2019

(54) LESION IDENTIFICATION MARKER UTILIZING BONE CEMENT FOR USE IN RADIATION THERAPY, AND LESION IDENTIFICATION MARKER KIT FOR USE IN RADIATION THERAPY

(71) Applicant: National University Corporation Hokkaido University, Sapporo-shi, Hokkaido (JP)

(72) Inventors: Daisuke Abo, Sapporo-shi, Hokkaido (JP); Yusuke Sakuhara, Sapporo-shi, Hokkaido (JP); Ryo Morita, Sapporo-shi, Hokkaido (JP); Naoki Miyamoto, Sapporo-shi, Hokkaido (JP)

(21) Appl. No.: 16/327,897

(22) PCT Filed: Aug. 24, 2017
(86) PCT No.: PCT/JP2017/030389

§ 371 Date: Feb. 25, 2019

(30) Foreign Application Priority Data
Aug. 25, 2016 (JP) 2016-164988

(51) Int. Cl. A61K 49/04; A61N 5/10

(52) U.S. Cl. CPC A61K 49/04; C01G 7/00; A61N 5/10

(57) ABSTRACT

Provided is a lesion identification marker for use in radiation therapy that enables pure gold microparticles which absorb X-rays to be placed, with extremely low invasive potential, in any site in the body in an arbitrary amount appropriate for the type of radiation therapy and the therapeutic target site, and that enables the placement site to be identified over a long period of time by radiation therapy equipment. The lesion identification marker for use in radiation therapy includes a mixture of pure gold particles and a substance containing a calcium phosphate-based bone reinforcing material, or a mixture of pure gold particles, a mixing solution, and a substance containing a calcium phosphate-based bone reinforcing material. The volume mean diameter (MV) of the particles of the substance containing a calcium phosphate-based bone reinforcing material is in the range of 3-12 μm.

### TECHNICAL FIELD

The present invention relates to a lesion identification marker for use in radiation therapy that is capable of being placed in various sites in the body via a thin puncture needle or the like, and relates to a lesion identification marker for use in radiation therapy and a lesion identification marker kit for use in radiation therapy that include fine pure gold particles and a substance containing a calcium phosphate-based bone reinforcing material.

### BACKGROUND

Technological developments such as intensity modulated radiation therapy (IMRT), image guided radiation therapy (IGRT), and real-time tumor-tracking radiation therapy (RTRT) have enabled high-precision radiation therapy in various organs including the lungs, the prostate, the liver, and the adrenal glands (see Non-Patent Document 1).

In these therapies, a metallic marker for lesion identification is embedded in an organ to acquire accurate location information of a target lesion by X-ray fluoroscopy (see Non-Patent Documents 1 and 2). The metallic marker becomes an indicator showing the location of a tumor in an X-ray fluoroscopic image, allowing efficient irradiation of the lesion with radiation while avoiding normal tissue to the extent possible. Consequently, therapeutic effects can be increased, while reducing the radiation dose toward the surrounding normal tissue and decreasing the risk of occurrence of adverse events.

Currently, markers for radiation therapy approved in Japan include iGold (a registered trademark) (Medikit Co., Ltd.), VISICOIL (Sceti Medical Labo KK), and Gold Anchor (Anzai Medical Co., Ltd.).

iGold is a 2 mm diameter sphere of pure gold, which has a high visibility by X-ray fluoroscopy, is capable of being recognized with the same shape from all directions, provides excellent accuracy when ascertaining location information, and further, is capable of being placed inside the trachea, the digestive tract, and the bladder mucosa (see Non-Patent Documents 2, 3, 4, 5, and 6). However, percutaneous placement requires the puncture of a 2.55 mm diameter sheath introducer (tube), and a safe puncture route cannot always be ensured depending on the site or the organ.

VISICOIL is a coil having a thin diameter (0.35 to 1.10 mm) and a length of 10 to 30 mm that is capable of being placed with a 19 G (1.10 mm) to 17 G (1.25 mm) needle, and the puncture route can be selected relatively easily (see Non-Patent Document 7). However, because VISICOIL is a coil having a thin diameter, it can sometimes not be recognized by X-ray fluoroscopy depending on the direction (especially the tangential direction), and location information cannot always be obtained unless placement of a plurality of coils is performed. Multiple punctures should be avoided because risks such as bleeding and organ damage are increased.

Gold Anchor is a gold wire processed by sawtooth cutting consisting of a gold alloy (iron content: 0.5 wt %) having a diameter of 0.28 mm and a length of 10 mm or 20 mm, is capable of being placed using a 22 G (outer diameter: 0.70 mm) to 25 G (outer diameter: 0.50 mm) needle, and the puncture route can be selected relatively easily. However, because Gold Anchor has a thin diameter, it can sometimes not be recognized by X-ray fluoroscopy depending on the direction (especially the tangential direction), and location information cannot always be obtained unless placement of a plurality of wires is performed. Although the wire is capable of being folded and used as a ball shape, an operation that inserts and ejects a needle within the organ is required during placement, which complicates the procedure. Risks such as bleeding and organ damage are also increased.

Furthermore, although Non-Patent Document 8 also describes an X-ray marker using gold nanoparticles, the visibility is insufficient because of the small amount of gold particles, and the visibility in locations where bone exists is also insufficient.

On the other hand, one disclosed technique using a mixture of calcium phosphate and a radiopaque material is a case where about 2 wt % of gold powder is mixed with calcium phosphate (see Patent Document 1), but for purposes as a lesion identification marker for use in radiation therapy, the visibility is insufficient, making the technique unusable. When a high concentration of gold particles and the like is included, the mixture of calcium phosphate and the radiopaque material tends to have a high viscosity, and is no longer capable of passing through a thin diameter needle, whereas if the viscosity is too low, it is essential to investigate conditions from a plurality of perspectives due to the difficulty of achieving a homogenous dispersion of relatively large gold particles having a high specific gravity and the like, and therefore it is not easy to find the optimal conditions. Furthermore, the use of extremely fine metal particles is recommended for metal and inorganic metal compounds capable of being used for improving the radiopacity, but in the case of pure gold particles having high purity, aggregation of the particles occurs particularly easily, and the use of fine gold particles is actually impractical.

Patent Documents 2 to 4 and Non-Patent Documents 9 and 10 describe various calcium phosphate-based compositions.

In Non-Patent Document 11, confirmation is made as to whether injection and placement of 0.1 to 0.2 mL of a paste-like mixture of pure gold particles having a particle width of 0.7±0.1 mm and 0.4±0.1 mm, a substance containing a calcium phosphate-based bone reinforcing material (sometimes referred to as a CPC below), and a dedicated mixing solution for the bone reinforcing material into a thawed, extracted pig liver using a short 18 G needle (needle length: about 3 to 4 cm) enables sufficient visibility to be obtained as a lesion identification marker for use in radiation therapy. Investigation of various changes in the proportion of pure gold particles in the pure gold particle/CPC mixture suggested that a mixture having a weight ratio of at least 1:4 (pure gold concentration: 20 wt %), or a mixture preferably having a weight ratio of at least 1:2 (pure gold concentration: 33 wt %), were preferable as a lesion identification marker for use in radiation therapy. However, passage of the pure gold particle/CPC paste of the above conditions through a long needle thinner than 18 G was not easy, and particularly in the case of the 0.7±0.1 mm diameter gold particles, the paste was sometimes unable to pass through even an 18 G needle, making the paste particularly unsuitable for use in puncture needles with a thin inner diameter (needle length: about 20 cm), or in endoscopes or the like.

### CITATION LIST

### Patent Literature

Patent Document 1: JP 2006-524058 A
Patent Document 2: JP S64-037445 A
Patent Document 3: JP 2002-255603 A
Patent Document 4: JP 2002-291866 A

### Non Patent Literature

Non-Patent Document 1: Dawson L A, Sharpe M B. Image-guided radiotherapy: rationale, benefits, and limitations. The lancet oncology. 2006; 7(10): 848-58.
Non-Patent Document 2: Shirato H, Shimizu S, Kunieda T, et al. Physical aspects of a real-time tumor-tracking system for gated radiotherapy. International journal of radiation oncology, biology, physics. 2000; 48(4): 1187-95.
Non-Patent Document 3: Shimizu S, Shirato H, Ogura S, et al. Detection of lung tumor movement in real-time tumor-tracking radiotherapy. International joumal of radiation oncology, biology, physics. 2001; 51(2): 304-10.
Non-Patent Document 4: Kitamura K, Shirato H, Seppenwoolde Y, et al. Three-dimensional intrafractional movement of prostate measured during real-time tumor-tracking radiotherapy in supine and prone treatment positions. International journal of radiation oncology, biology, physics. 2002; 53(5): 1117-23.
Non-Patent Document 5: Taguchi H, Sakuhara Y, Hige S, et al. Intercepting radiotherapy using a real-time tumor-tracking radiotherapy system for highly selected patients with hepatocellular carcinoma unresectable with other modalities. Intemational journal of radiation oncology, biology, physics. 2007; 69(2): 376-80.
Non-Patent Document 6: Katoh N, Onimaru R, Sakuhara Y, et al. Real-time tumor-tracking radiotherapy for adrenal tumors. Radiotherapy and oncology: journal of the European Society for Therapeutic Radiology and Oncology. 2008; 87(3): 418-24.
Non-Patent Document 7: Kim J H, Hong S S, Kim J H, et al. Safety and efficacy of ultrasound-guided fiducial marker implantation for CyberKnife radiation therapy. Korean journal of radiology: official journal of the Korean Radiological Society. 2012; 13(3): 307-13.
Non-Patent Document 8: Adv. Healthcare Mater., 2015, 4, p. 856-863
Non-Patent Document 9: Journal of the Ceramic Society of Japan, Vol. 84(4), 1976, p. 209-213
Non-Patent Document 10: J. Soc. Inorganic Mat. Jap., Vol. 12, p. 262-269 (2005)
Non-Patent Document 11: Hokkaido Angiography and Interventional Radiology Research Meeting, Aug. 8, 2015

### SUMMARY

### Technical Problem

Future lesion identification markers for use in radiation therapy will be required to: (1) be a highly biocompatible substrate; (2) enable placement using a thin puncture needle (20 G to 22 G (outer diameter: about 0.9 to 0.7 mm; inner diameter: about 0.7 to 0.5 mm)) in view of reducing patient burden, and broadening application (capable of being placed in a variety of organs and tissues) and the like; (3) ensure visibility (ease of viewing the marker within an image), image recognition performance, and tracking performance with an arbitrary placement amount of gold particles appropriate for the site; (4) display rapid solidification (shortening of treatment times) and have a high shape retention in view of reducing elimination and migration (capable of being used over a long period); and the like (see FIG. 1).

An object of the present invention is to provide a lesion identification marker for use in radiation therapy and a lesion identification marker kit for use in radiation therapy that enable pure gold microparticles which absorb X-rays to be placed, with extremely low invasive potential, in any site in the body in an arbitrary amount appropriate for the type of radiation therapy and the therapeutic target site, and that enable the placement site to be identified over a long period of time by radiation therapy equipment.

### Solution to Problem

The present invention provides a lesion identification marker for use in radiation therapy that includes either a mixture of pure gold particles and a substance containing a calcium phosphate-based bone reinforcing material, or a mixture of pure gold particles, a mixing solution, and a substance containing a calcium phosphate-based bone reinforcing material, wherein a volume mean diameter (MV) of the particles of the substance containing a calcium phosphate-based bone reinforcing material is within a range from 3 to 12 μm.

In the lesion identification marker for use in radiation therapy, a kneaded material obtained from the mixing solution and the substance containing a calcium phosphate-based bone reinforcing material preferably has a viscosity at 20° C. within a range from 10⁸ to 10¹⁰ mPa·s about 5 min after the start of kneading.

In the lesion identification marker for use in radiation therapy, a median diameter (D50) of the pure gold particles is preferably within a range from 16 to 40 μm.

In the lesion identification marker for use in radiation therapy, a median diameter (D50) of the pure gold particles is more preferably within a range from 20 to 35 μm.

In the lesion identification marker for use in radiation therapy, the pure gold particles preferably have a D10 of at least 5 μm, and a D90 of 70 μm or less.

In the lesion identification marker for use in radiation therapy, the pure gold particles more preferably have a D10 of at least 10 μm, and a D90 of 55 μm or less.

In the lesion identification marker for use in radiation therapy, a volume mean diameter (MV) of the pure gold particle is preferably within a range from 17 to 44 μm.

In the lesion identification marker for use in radiation therapy, a volume mean diameter (MV) of the pure gold particles is more preferably within a range from 20 to 38 μm.

In the lesion identification marker for use in radiation therapy, D90 of the particles of the substance containing a calcium phosphate-based bone reinforcing material is preferably less than 39 μm.

In the lesion identification marker for use in radiation therapy, D90 of the particles of the substance containing a calcium phosphate-based bone reinforcing material is more preferably within a range from 10 to 30 μm.

In the lesion identification marker for use in radiation therapy, the pure gold particles preferably have an abundance ratio of less than about 3% (volume ratio) for particles having a particle size exceeding about 96 μm.

In the lesion identification marker for use in radiation therapy, the pure gold particles more preferably have an abundance ratio of less than about 1.5% (volume ratio) for particles having a particle size exceeding about 96 μm.

In the lesion identification marker for use in radiation therapy, the substance containing a calcium phosphate-based bone reinforcing material preferably has an abundance ratio of less than about 15% (volume ratio) for particles having a particle size exceeding about 31 μm.

In the lesion identification marker for use in radiation therapy, the mixture is preferably capable of passing through a 20 G to 22 G puncture needle with a needle length of 20 cm.

In the lesion identification marker for use in radiation therapy, a mixing ratio by volume of the mixing solution per gram of the substance containing a calcium phosphate-based bone reinforcing material is preferably within a range from about 0.3 mL/g to 0.5 mL/g.

In the lesion identification marker for use in radiation therapy, a weight ratio between the pure gold particles and the substance containing a calcium phosphate-based bone reinforcing material is preferably at least 1:2 but not more than 2:1.

In the lesion identification marker for use in radiation therapy, the pure gold particles are preferably pure gold particles having a purity of at least 99 wt %.

In the lesion identification marker for use in radiation therapy, at least 5 mg of the pure gold particles are preferably included.

In the lesion identification marker for use in radiation therapy, the substance containing a calcium phosphate-based bone reinforcing material preferably contains at least one of α-tricalcium phosphate, tetracalcium phosphate, calcium hydrogen phosphate (anhydride or hydrate), and β-tricalcium phosphate.

In the lesion identification marker for use in radiation therapy, the mixing solution is preferably at least one of a mixing solution containing sodium chondroitin sulfate ester, disodium succinate anhydride, sodium bisulfite, and water, and a mixing solution containing dextran sulfate ester sodium sulfur 5 and water.

Furthermore, the present invention is a lesion identification marker kit for use in radiation therapy including pure gold particles and a substance containing a calcium phosphate-based bone reinforcing material, or pure gold particles, a mixing solution, and a substance containing a calcium phosphate-based bone reinforcing material, wherein

a volume mean diameter (MV) of the particles of the substance containing a calcium phosphate-based bone reinforcing material is within a range from 3 to 12 μm.

In the lesion identification marker kit for use in radiation therapy, a kneaded material obtained from the pure gold particles, the substance containing a calcium phosphate-based bone reinforcing material and the mixing solution preferably has a viscosity at 20° C. of 10⁸ to 10¹⁰ mPa·s about 5 min after the start of kneading.

In the lesion identification marker kit for use in radiation therapy, a median diameter (D50) of the pure gold particles is preferably within a range from 16 to 40 μm.

In the lesion identification marker kit for use in radiation therapy, a median diameter (D50) of the pure gold particles is more preferably within a range from 20 to 35 μm.

In the lesion identification marker kit for use in radiation therapy, the pure gold particles preferably have a D10 of at least 5 μm, and a D90 of 70 μm or less.

In the lesion identification marker kit for use in radiation therapy, the pure gold particles more preferably have a D10 of at least 10 μm, and a D90 of 55 μm or less.

In the lesion identification marker kit for use in radiation therapy, a volume mean diameter (MV) of the pure gold particle is preferably within a range from 17 to 44 μm.

In the lesion identification marker kit for use in radiation therapy, a volume mean diameter (MV) of the pure gold particles is more preferably within a range from 20 to 38 μm.

In the lesion identification marker kit for use in radiation therapy, a volume mean diameter (MV) of the particles of the substance containing a calcium phosphate-based bone reinforcing material is preferably within a range from 3 to 12 μm.

In the lesion identification marker kit for use in radiation therapy, D90 of the particles of the substance containing a calcium phosphate-based bone reinforcing material is preferably less than 39 μm.

In the lesion identification marker kit for use in radiation therapy, D90 of the particles of the substance containing a calcium phosphate-based bone reinforcing material is more preferably within a range from 10 to 30 μm.

In the lesion identification marker kit for use in radiation therapy, the pure gold particles preferably have an abundance ratio of less than about 3% (volume ratio) for particles having a particle size exceeding about 96 μm.

In the lesion identification marker kit for use in radiation therapy, the pure gold particles more preferably have an abundance ratio of less than about 1.5% (volume ratio) for particles having a particle width exceeding about 96 μm.

In the lesion identification marker kit for use in radiation therapy, the substance containing a calcium phosphate-based bone reinforcing material preferably has an abundance ratio of less than about 15% (volume ratio) for particles having a particle width exceeding about 31 μm.

In the lesion identification marker kit for use in radiation therapy, a kneaded material obtained from the pure gold particles and the substance containing a calcium phosphate-based bone reinforcing material, or a kneaded material obtained from the pure gold particles, the mixing solution, and the substance containing a calcium phosphate-based bone reinforcing material, is preferably capable of passing through a 20 G to 22 G puncture needle with a needle length of 20 cm.

In the lesion identification marker kit for use in radiation therapy, a mixing ratio by volume of the mixing solution per gram of the substance containing a calcium phosphate-based bone reinforcing material is preferably within a range from about 0.3 mL/g to 0.5 mL/g.

In the lesion identification marker kit for use in radiation therapy, a weight ratio between the pure gold particles and the substance containing a calcium phosphate-based bone reinforcing material is preferably at least 1:2 but not more than 2:1.

In the lesion identification marker kit for use in radiation therapy, the pure gold particles are preferably pure gold particles having a purity of at least 99 wt %.

In the lesion identification marker kit for use in radiation therapy, at least 5 mg of the pure gold particles are preferably included.

In the lesion identification marker kit for use in radiation therapy, the substance containing a calcium phosphate-based bone reinforcing material preferably contains at least one of α-tricalcium phosphate, tetracalcium phosphate, calcium hydrogen phosphate (anhydride or hydrate), and β-tricalcium phosphate.

In the lesion identification marker kit for use in radiation therapy, the mixing solution is preferably at least one of a mixing solution containing sodium chondroitin sulfate ester, disodium succinate anhydride, sodium bisulfite, and water, and a mixing solution containing dextran sulfate ester sodium sulfur 5 and water.

### Advantageous Effects of Invention

According to the present invention, a lesion identification marker for use in radiation therapy and a lesion identification marker kit for use in radiation therapy can be provided that enable pure gold microparticles which absorb X-rays to be placed, with extremely low invasive potential, in any site of the body in an arbitrary amount appropriate for the type of radiation therapy and the therapeutic target site, and that enable the placement site to be identified over a long period of time by radiation therapy equipment.

### BRIEF DESCRIPTION OF DRAWINGS

FIG. 1 is an image of a lesion identification marker for use in radiation therapy disclosed in the present invention.

FIG. 2 are diagrams showing a flat panel detector used in evaluating the image recognition performance of a G/CPC marker of the present invention.

FIG. 3 is an X-ray fluoroscopic image in Example 4, where a variety of weights of G/CPC marker clusters having weight concentrations of pure gold particles of 33 wt %, 66 wt %, and 80 wt % have been placed in a 96-well plate in order to investigate the relationship between the pure gold concentration (wt %) in the G/CPC mixture and the image recognition performance (acrylic plate thickness: 1 cm; tube current: 50 mA; tube voltage: 110 kV; irradiation time: 3 msec).

FIG. 4 is a scanning electron microscope (SEM) photograph (magnification: 500×) in Example 7, showing pure gold particles having a particle width of 75 to 54 μm.

FIG. 5 is a scanning electron microscope (SEM) photograph (magnification: 10,000×) in Example 7, showing gold particles manufactured by The Nilaco Corporation having a particle size of 1 to 2 μm (after grinding in a mortar).

FIG. 6 represents graphs of the particle size distributions of the various pure gold particles measured by a particle size distribution measurement device in Example 10, wherein the horizontal axis (X axis) indicates the particle size, and the vertical axis (Y axis) indicates the frequency (%) of particles of each particle size. The graphs show the particle size distributions of pure gold particles having (a) a particle width of 20 μm or less; (b) a particle width of 53 to 33 μm; (c) a particle width of 32 μm or less; and (d) a particle width of 75 to 54 μm.

FIG. 7 represents graphs of the particle size distributions of gold particles manufactured by The Nilaco Corporation measured by a particle size distribution measurement device in Example 10, wherein (a) is a graph of the particle size distribution of a gold particle powder obtained after grinding gold particles (commercially available product) manufactured by The Nilaco Corporation with an agate mortar; and (b) is a graph of the particle size distribution of a fraction of gold particles obtained after sieving the mortar-ground product using a sieve having 32 μm openings.

FIG. 8 represents graphs of the particle size distributions of the various pure CPC powders measured by a particle size distribution measurement device in Example 11, wherein the horizontal axis (X axis) represents the particle size, and the vertical axis (Y axis) represents the frequency (%) of particles of each particle size. The graphs show the particle size distributions of (a) the commercially available product Biopex-R Excellent Type; (b) the commercially available product Biopex-R Long Type; (c) a fraction of the powder obtained after sieving the commercially available product Biopex-R Excellent Type using a sieve having 32 μm openings; and (d) a fraction of the powder obtained after sieving the commercially available product Biopex-R Long Type using a sieve having 32 μm openings.

FIG. 9 is a photograph showing a partially opened dog abdomen at the time of injection of a G/CPC paste into the liver site.

FIG. 10 is an X-ray fluoroscopic image in Example 12, showing a partial lobe of a dog liver, extracted 28 days after injection of a G/CPC paste (acrylic plate thickness: 1 cm). In the diagram, (1) to (5) are markers placed on the liver surface as positive controls ((1): 1.5 mm diameter gold spheres; (2): 2.0 mm diameter gold spheres; (3): 0.28×10 mm Gold Anchor, compressed; (4): 0.28×20 mm Gold Anchor, compressed; and (5): 0.28×20 mm Gold Anchor, linear (tracking the tip portion)), and in the diagram, (6) to (10) are G/CPC markers.

FIG. 11 is a diagram showing the change in laboratory test values in a dog observed for 28 days after injection of a G/CPC paste in Example 12.

FIG. 12 is a graph comparing the viscosity transitions of kneaded materials of Biopex-R Excellent, Biopex-R Standard, and Long.

FIG. 13 is a graph comparing the viscosity transitions of kneaded materials of the commercially available product and sieved products of Biopex-R Excellent.

FIG. 14 is a graph comparing the viscosity transitions of kneaded materials of sieved products of Biopex-R Excellent (32 μm sieve) from differences in the gold particle content.

FIG. 15 is a photograph showing an X-ray fluoroscopic image of five G/CPC markers 28 days after placement in the liver of a live dog.

FIG. 16 is a photograph showing an X-ray fluoroscopic image of six G/CPC markers 28 days after placement in the pancreas of a live dog.

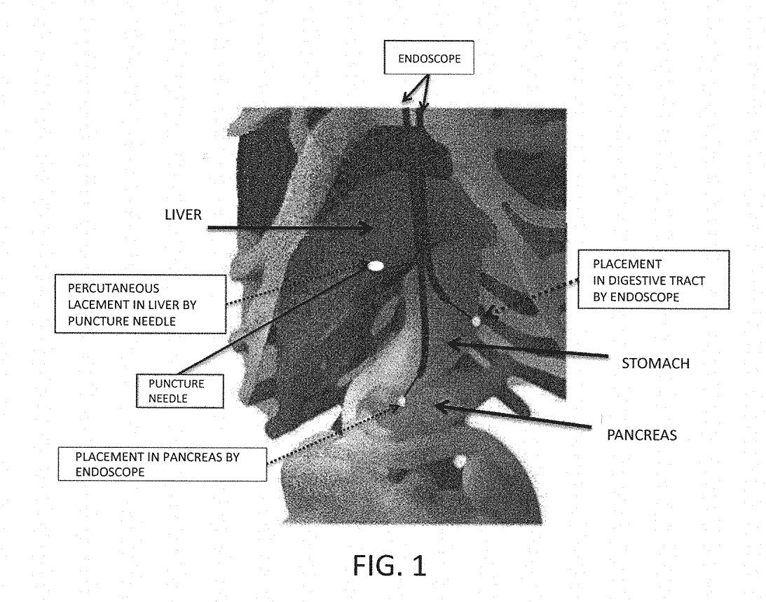

### DESCRIPTION OF EMBODIMENTS

Embodiments of the present invention are described below. These embodiments are merely examples of implementing the present invention, and the present invention is in no way limited by these embodiments.

### <Lesion Identification Marker for Use in Radiation Therapy>

A lesion identification marker for use in radiation therapy according to an embodiment of the present invention includes either a mixture of pure gold particles and a substance containing a calcium phosphate-based bone reinforcing material, or a mixture of pure gold particles, a mixing solution, and a substance containing a calcium phosphate-based bone reinforcing material. The lesion identification marker for use in radiation therapy according to an embodiment of the present invention includes a mixture of pure gold particles and a substance containing a calcium phosphate-based bone reinforcing material having a volume mean diameter (MV) within a range from 3 to 12 μm. The lesion identification marker for use in radiation therapy according to an embodiment of the present invention preferably includes a mixture of pure gold particles having a median diameter (D50, volume basis) of 16 to 40 μm, and a substance containing a calcium phosphate-based bone reinforcing material having a volume mean diameter (MV) within a range from 3 to 12 μm, or more preferably includes a mixture of pure gold particles having a median diameter (D50, volume basis) of 20 to 35 μm, and a substance containing a calcium phosphate-based bone reinforcing material having a volume mean diameter (MV) within a range from 3 to 12 μm. Furthermore, the lesion identification marker for use in radiation therapy according to an embodiment of the present invention includes a mixture of pure gold particles, a mixing solution, and a substance containing a calcium phosphate-based bone reinforcing material having a volume mean diameter (MV) within a range from 3 to 12 μm. The lesion identification marker for use in radiation therapy according to an embodiment of the present invention preferably includes a mixture of pure gold particles having a median diameter (D50, volume basis) of 16 to 40 μm, a mixing solution, and a substance containing a calcium phosphate-based bone reinforcing material having a volume mean diameter (MV) within a range from 3 to 12 μm, or more preferably includes a mixture of pure gold particles having a median diameter (D50, volume basis) of 20 to 35 μm, a mixing solution, and a substance containing a calcium phosphate-based bone reinforcing material having a volume mean diameter (MV) within a range from 3 to 12 μm.

The lesion identification marker for use in radiation therapy according to the present embodiment enables either a mixture of pure gold particles and a substance containing a calcium phosphate-based bone reinforcing material, or a mixture of pure gold particles, a mixing solution, and a substance containing a calcium phosphate-based bone reinforcing material, to be placed and embedded by a puncture needle or the like, in any site in the body. The lesion identification marker for use in radiation therapy according to the present embodiment enables pure gold (purity: at least 99 wt %) microparticles, which absorb X-rays and have a high biocompatibility, to be placed in any site in the body in an arbitrary amount appropriate for the type of radiation therapy and the therapeutic target site, and because placement is possible using a 20 G to 22 G (outer diameter: about 0.9 to 0.7 mm inner diameter: about 0.7 to 0.5 mm) puncture needle with a needle length of 20 cm, the invasive potential can be significantly improved compared to the presently used iGold (in which 2.0 mm diameter particles are placed using an introducer having an outer diameter of 2.6 mm), and enables the placement site to be identified over a long period of time by radiation therapy equipment. In other words, the lesion identification marker for use in radiation therapy according to the present embodiment allows notable effects to be obtained, including: (1) a highly biocompatible substrate; (2) enabling placement using, for example, a thin puncture needle (20 G to 22 G (outer diameter: about 0.9 to 0.7 mm; inner diameter: about 0.7 to 0.5 mm)) with a needle length of 5 cm to 20 cm in view of reducing patient burden, and broadening application (capable of being placed in a variety of organs and tissues) and the like; (3) ensuring visibility (ease of viewing the marker within an image), image recognition performance, and tracking performance appropriate for the site; (4) displaying rapid solidification (shortening of treatment times) and having a high shape retention in view of reducing elimination and migration (capable of being used over a long period); and the like. In the present description, evidence is presented using pure (purity: at least 99.99 wt %) microparticles, but substances in which a gold alloy (for example, a highly biocompatible metal having a gold content of at least 99 wt %), a mixing solution, and a substance containing a calcium phosphate-based bone reinforcing material are mixed and kneaded together are also capable of being used as a lesion identification marker for use in radiation therapy. In the present description, the visibility, the image recognition performance, and the tracking performance are used with the following types of meanings.

1) Visibility: a qualitative evaluation of the ease of viewing the marker within an image as determined by the human eye

2) Image recognition performance: a quantitative evaluation of the image recognition performance of the marker as a numerical value using pattern matching

3) Tracking performance: an evaluation of whether or not a moving marker can be tracked using pattern matching

The lesion identification marker for use in radiation therapy according to the present invention enables pure gold or gold alloy (for example, at least 99 wt % of gold, and less than 1 wt % of another metal) microparticles which absorb X-rays to be placed in any site in the body in an arbitrary amount appropriate for the type of radiation therapy and the therapeutic target site, and enables the placement site to be identified by radiation therapy equipment. For example, the marker is capable of passing through a 20 G to 22 G puncture needle with a needle length of 20 cm, enables placement by delivery systems that were conventionally unusable, and can have visibility comparable to, or better than, conventional 2 mm diameter pure gold markers. Sufficient shape retention, tracking performance (whether or not a moving marker can be tracked), and safety are suggested when placed inside an animal body, and as a lesion identification marker capable of being safely placed in any site (various organs, and tissues and the like) in the body, the marker is capable of further expanding the applications of high-precision radiation therapy, and enables development of pathways to new cancer therapies. In contrast to conventional metallic markers, because the lesion identification marker for use in radiation therapy according to the present embodiment is a paste, the placement amount can be arbitrarily selected according to the placement target site and the target patient to ensure favorable visibility and tracking performance, even under different conditions. For example, it is possible to place a small amount of the paste in children, and a large amount in sites that have a low X-ray transparency in overweight adults, which may be stated as a significant advantage in comparison to conventional metallic markers. Because the lesion identification marker for use in radiation therapy according to the present embodiment can be placed with a much thinner puncture instrument than is conventionally possible, the risk of bleeding caused by the puncture at the time of placement may be low, and patient burden, such as organ disorders, may be extremely low. Furthermore, because placement can be achieved by a thin tube such as a catheter, it is possible to perform placement from inside the digestive tract, trachea, or bronchial tube using an endoscope, placement inside the bladder via the urethra, placement inside the uterus via the vagina and the like, and there is also a clear advantage over conventional methods in terms of the invasive potential.

Methods for placing the lesion identification marker according to an embodiment of the present invention in any site in the body include, for example, a method that includes: mounting a porous body such as a collagen sponge on the tip of a placement needle and preparing a substance in which the porous body is filled with pure gold particles, attaching a syringe that is filled with an appropriate amount of a kneaded material in which a mixing solution and a substance containing a calcium phosphate-based bone reinforcing material are mixed (calcium phosphate-based bone reinforcing material kneaded material), and pushing the pure gold particle clusters into an organ using the calcium phosphate-based bone reinforcing material kneaded material; and a method that includes: producing a pure gold particle-containing calcium phosphate-based bone reinforcing material kneaded material by mixing pure gold particles, a mixing solution, and a substance containing a calcium phosphate-based bone reinforcing material, transferring the kneaded material to a syringe, attaching a needle, and injecting the lesion identification marker containing the pure gold particles and the substance containing a calcium phosphate-based bone reinforcing material into an organ.

The method that pushes the pure gold particle clusters using a calcium phosphate-based bone reinforcing material kneaded material or the like has the advantages that high visibility can be achieved with a small amount of pure gold particles, and that an operation for mixing the pure gold particles and the calcium phosphate-based bone reinforcing material kneaded material can be unnecessary.

In the method that uses a pure gold particle-containing calcium phosphate-based bone reinforcing material kneaded material, because the material is in a paste form at the time of injection, injection is possible from not only a needle, but also a catheter or the like, enabling a lesion identification marker that includes pure gold particles and a substance containing a calcium phosphate-based bone reinforcing material to also be placed in the gastrointestinal mucosa, the pancreas, or the bladder or the like, via a catheter.

Pure gold particles have a high biocompatibility and a good visibility. The pure gold particles are preferably pure gold particles having a purity of at least 99 wt %, more preferably pure gold particles having a purity of at least 99.9 wt %, and particularly preferably pure gold particles having a purity of at least 99.99 wt %.

Pure gold particles have a spherical shape or an amorphous shape or the like, and although smaller particle widths are generally considered to be better, aggregation between the pure gold particles is observed if the particle size is too small, and particularly in products having a particle size of 1 to 2 μm (manufactured by The Nilaco Corporation), particle clusters of at least several hundred μm are also included. The same phenomenon is also observed in gold particles that have been fractionated by a sieve after preparation by the atomizer method described below, and at least in the case of pure gold particles, the notion that smaller particles are better was found to be inappropriate, and ultimately, determinations were made based on the passability through a clinically-used puncture needle with a needle length of 20 cm. As a result, it was shown that pure gold particles having a particle width (as defined by openings of the sieve used) of 53 to 33 μm, and 32 μm or less are preferable, and pure gold particles having a particle width of 32 μm or less are more preferable. In pure gold particles manufactured by The Nilaco Corporation (product number: AU-174015), it was also confirmed that a fraction of gold microparticles sieved by a 32 μm sieve passed through a 21 to 22 G puncture needle in a similar manner to that described above. Although the particle width of pure gold particles is, as described in (A) below, determined based on passage or non-passage through a sieve having prescribed openings (JIS Z 8801), the particle width may be more appropriately defined by a median diameter (D50), or by D10 or D90 or the like, by using the measurement results of a particle size distribution measurement.

When the particle size distribution is represented by a cumulative distribution, the side of the distribution representing the finest particles is expressed as zero, and D50, D10, and D90 and the like are used to represent the distribution. The D50 value is referred to as a median diameter, and represents the diameter at which the larger side and the smaller side are equal, while D10 is the particle size at which the cumulative distribution from the side representing the smallest particles is 10%, and D90 is the particle size at which the cumulative distribution from the side representing the largest particles is 10%. MV represents the volume mean diameter, MN the number average diameter, and MA the area average diameter.

From the perspective of the passability through a long, thin diameter needle, the D50 value of the pure gold particles is preferably within a range from 16 to 40 μm, more preferably within a range from 18 to 36 μm, even more preferably within a range from 20 to 35 μm, and particularly preferably within a range from 20 to 32 μm. Further, for a D50 value within the above range, a D10 value of at least 5 μm and a D90 value of 70 μm or less are preferred, a D10 value of at least 7 μm and a D90 value of 60 μm or less are more preferred, and a D10 value of at least 10 μm and a D90 value of 55 μm or less are even more preferred. If the D50 value of the pure gold particles exceeds about 40 μm, passage through a 20 G to 22 G puncture needle with a needle length of 20 cm may sometimes become difficult.

Furthermore, using the volume mean diameter (MV) to express the preferred pure gold particles, a range from 17 to 44 μm is preferred, a range from 17 to 38 μm is more preferred, and a range from 20 to 38 μm is even more preferred. If the MV of the pure gold particles exceeds about 45 μm, passage through a 20 G to 22 G puncture needle with a needle length of 20 cm may sometimes become difficult.

Moreover, in the pure gold particles, it is preferable that the abundance ratio (volume ratio) of particles having a particle size exceeding about 96 μm (for example, 95.96 μm) is less than about 3%, more preferably about 2.5% or less, and particularly preferably about 1.5% or less. In the pure gold particles, if the abundance ratio of particles having a particle size exceeding about 96 μm exceeds about 3%, passage through a 20 G to 22 G puncture needle with a needle length of 20 cm may sometimes become difficult. The cumulative frequency (volume distribution) of particles in which the particle size is about 40 μm or less (for example, 40.35 μm) is preferably at least 50%, more preferably at least 65%, even more preferably at least 70%, and particularly preferably at least 85%, while the cumulative frequency up to a particle size of about 31 μm (for example, 31.11 μm) is preferably at least 30%, more preferably at least 35%, and even more preferably at least 70%.

In the present description, gold particles produced by the method described below are mainly used, and these particles are respectively referred to as pure gold particles having a particle width of 150 to 76 μm, 75 to 54 μm, 53 to 33 μm, 32 μm or less, and 20 μm or less.

(A) An outline of the production method of the pure gold particles is presented below.

(1) Pure gold particles are prepared by spraying a heated melt of pure gold (at least 99.99 wt %) from an atomizer having at least one of a carbon nozzle and a quartz nozzle, followed by rapid cooling.

(2) The particles are sequentially sieved using sieves with openings (JIS Z 8801) of 150 μm, 75 μm, 53 μm, and 32 μm to obtain pure gold particles having the respective particle widths of 150 to 76 μm, 75 to 54 μm, 53 to 33 μm, and 32 μm or less. Further, the pure gold particles having a particle width of 32 μm or less are sieved using a 20 μm sieve to obtain pure gold particles having a particle width of 20 μm or less. The sieving may be performed using a manual sieving method using tapping balls such as polyurethane balls with an iron core, or a method using a sonic sieve.

The particle size distributions of the respective pure gold particles are measured by a wet method using a particle size distribution measurement device manufactured by MicrotracBEL Corporation (MT3000II) and IPA (isopropyl alcohol) as the dispersion medium, and in addition to determining the D50 (medium diameter), the D10 the D90, the MV (volume mean diameter), the MN (number average diameter) and the MA (area average diameter) of the particle size distributions, the particle size distributions of the pure gold particles are also displayed as a graph. Theoretically, the measurement method of the above equipment measures the volume distribution, and a volume basis is used as the basis for the abundance ratio of the particles. Furthermore, when the particle size distribution is represented by a cumulative distribution, the side representing the finest particles is expressed as zero.

When a mixture of pure gold particles and a substance containing a calcium phosphate-based bone reinforcing material, or a mixture of pure gold particles, a mixing solution, and a substance containing a calcium phosphate-based bone reinforcing material is placed as a lesion identification marker in any site in the body, in terms of functioning favorably as an X-ray marker, it is preferable for the weight of the included pure gold particles to be least about 5 mg, and more preferable for the weight of the included pure gold particles to be at least 20 mg.

The substance containing a calcium phosphate-based bone reinforcing material is a calcium phosphate-based composition, and examples of known substances include those having α-tricalcium phosphate (for example, see JP 2002-255603 A), tetracalcium phosphate (for example, see JP 2002-291866 A), calcium hydrogen phosphate (for example, see JP S64-037445 A), or β-tricalcium phosphate (for example, see JP 2010-075247 A) as the main ingredient. These calcium phosphate-based bone reinforcing materials are believed to be converted into hydroxyapatite in the body, the chemical formula of which is represented by Ca₁₀(PO₄)₆(OH)₂.

Calcium phosphate-based bone reinforcing materials are also visible under X-ray fluoroscopy, but because it is difficult to ensure the required visibility under X-ray fluoroscopy at the time of radiation therapy, sufficient visibility under X-ray fluoroscopy can be ensured by using a lesion identification marker in which a sufficient amount of pure gold particles, such as pure gold particles that have a high biocompatibility, is mixed as homogeneously as possible with the substance containing a calcium phosphate-based bone reinforcing material.

The substance containing a calcium phosphate-based bone reinforcing material includes, for example, at least one of α-tricalcium phosphate, tetracalcium phosphate, calcium hydrogen phosphate (anhydride or hydrate), and 3-tricalcium phosphate, and may additionally include at least one substance selected from among phosphate compounds such as hydroxyapatite, magnesium phosphate, amorphous calcium phosphate and calcium phosphate-based glass, polysaccharides, collagen, calcium phosphate/collagen composite, bone morphogenetic protein (BMP), and insulin-like growth factor (IGF).

The substance containing a calcium phosphate-based bone reinforcing material is, for example, in a powder form. The substance containing a calcium phosphate-based bone reinforcing material may use, for example, Biopex-R (Standard Type, Long Type, and Excellent Type) (manufactured by HOYA Technosurgical Corporation) which includes α-tricalcium phosphate (75 wt %), tetracalcium phosphate (18 wt %), calcium hydrogen phosphate (5 wt %), hydroxvapatite (2 wt %), and magnesium phosphate, or Cerapaste (manufactured by NGK Spark Plug Co., Ltd.) which is a mixed composite of tetracalcium phosphate and anhydrous calcium hydrogen phosphate.

The substance containing a calcium phosphate-based bone reinforcing material (CPC) is also sequentially sieved using sieves with openings (JIS Z 8801) of 75 μm, 53 μm, and 32 μm, and these are respectively referred to as CPCs having a particle width of at least 75 μm, 75 to 54 μm, 53 to 33 μm, and 32 μm or less. Furthermore, a CPC is also sometimes simply prepared by fractionation using individual sieves respectively having openings of 150 μm, 100 μm, 75 μm, 53 μm, 32 μm, 25 μm, and 20 μm. Therefore, for example, a particle width indicated to be 53 μm or less refers to a case where a fraction has been passed through a sieve with openings of 53 μm.

Among the CPCs, the particle size distributions of the commercially available product and those having a particle width of 75 μm, 53 μm, 32 μm, 25 μm, and 20 μm or less are measured by a wet method using a particle size distribution measurement device Microtrac MT3300EX-II (MicrotracBEL Corporation) and water as the dispersion medium, and in addition to determining the D50 (median diameter), the D10, the D90, the MV (volume mean diameter), the MN (number average diameter) and the MA (area average diameter) of the particle distributions, the particle size distributions of the CPCs are also displayed as graphs. In this case, in a similar manner to described above, the measurement theoretically measures a volume distribution, and a volume basis is used as the basis for the abundance ratio of the particles. Furthermore, when the particle size distribution is represented by a cumulative distribution, the side representing the finest particles is expressed as zero.

The particle width of the substance containing a calcium phosphate-based bone reinforcing material is preferably 75 μm or less, 53 to 33 μm or less, 53 μm or less, 32 μm or less, and 25 μm or less, more preferably 53 μm or less, 32 μm or less, and 25 μm or less, and even more preferably 32 μm or less and 25 μm or less, while the volume mean diameter (MV) of the particles is preferably within a range from 3 to 12 μm, more preferably within a range from 4 to 12 μm, even more preferably within a range from 5 to 11 μm, and particularly preferably within a range from 5 to 10 μm. Further, the D90 value of the particles of the substance containing a calcium phosphate-based bone reinforcing material is preferably less than 39 μm, more preferably 34 μm or less, even more preferably 30 μm or less, and particularly preferably within a range from 10 to 30 μm. On the other hand, if the D90 value exceeds 50 μm, passage through a 19 G to 22 G puncture needle with a needle length of 20 cm may sometimes become difficult.

Furthermore, in the substance containing a calcium phosphate-based bone reinforcing material, if the CPC particles having favorable passability through a thin puncture needle are expressed in terms of the particle size distribution exceeding about 31 μm (for example, 31.11 μm), then the distribution (frequency) of particles exceeding about 31 μm is preferably about 15% or less, and more preferably about 10% or less. In the particle size distribution of the substance containing a calcium phosphate-based bone reinforcing material, if the abundance ratio (volume ratio) of particles exceeding about 31 μm is more than 15%, passage of the G/CPC paste through a 19 G to 22 G puncture needle with a needle length of 20 cm may sometimes become difficult.

There are no particular limitations on the mixing solution for preparing a kneaded material of a substance containing a calcium phosphate-based bone reinforcing material (calcium phosphate-based bone reinforcing kneaded material), or the mixing solution for kneading together pure gold particles and a substance containing a calcium phosphate-based bone reinforcing material to prepare a pure gold particle-containing calcium phosphate-based bone reinforcing kneaded material, provided the solution can be used as a mixing solution of a calcium phosphate-based bone reinforcing material, and examples include a mixing solution that uses water or an acid (for example, hydrochloric acid, sulfuric acid, phosphoric acid, formic acid, acetic acid, succinic acid, or lactic acid or the like) in a mixture of one or more of a readily soluble halogen compound, a sulfate salt, and an organic acid salt (see JP S59-88351 A), a mixing solution that uses an acidic solution containing a homopolymer or copolymer of an unsaturated carboxylic acid (for example, acrylic acid, maleic acid, fumaric acid, or itaconic acid) (see JP S60-253454 A), a mixing solution containing an antimicrobial agent (for example, propylene glycol, or ethylene glycol or the like) and a water-soluble polymer (for example, chitin, chitosan, soluble starch, chondroitin sulfate or salts thereof, or carboxymethyl cellulose or the like) (see JP H03-267067), and a mixing solution containing a water-soluble sodium salt such as sodium succinate (see JP H04-12044 A). Examples of mixing solutions that may be used favorably include a mixing solution that combines the above and includes sodium chondroitin sulfate ester (sodium chondroitin sulfate), disodium succinate anhydride, sodium bisulfite, and water such as water for injection (Japanese Pharmacopoeia) (see JP 2002-255603 A), a mixing solution containing dextran sulfate ester sodium sulfur 5 (dextran sulfate sodium sulfur 5) and water such as water for injection (see JP 2002-291866 A), a mixing solution consisting of water such as water for injection, a mixing solution containing a water-soluble sodium salt such as sodium phosphate, and a mixing solution containing various organic acids such as citric acid or the like. In terms of the mixing solution, for example, a dedicated mixing solution for Biopex-R (manufactured by HOYA Technosurgical Corporation) containing disodium succinate anhydride (12 wt %), sodium chondroitin sulfate ester (5 wt %), sodium bisulfite, and water for injection (83 wt %), or a Cerapaste curing liquid (composition: dextran sulfate ester sodium sulfur 5 and water for injection) or the like may be used. Furthermore, although the commercially available mixing solutions may be used after dilution with an appropriate amount of water for injection, if water for injection alone is used as the mixing solution, blockage of the syringe and the like can sometimes occur at the time of injection of the kneaded material, or solidification of the kneaded material can sometimes require too much time.

In terms of the liquid amount of the mixing solution used for preparing a kneaded material of pure gold particles and a substance containing a calcium phosphate-based bone reinforcing material (calcium phosphate-based bone reinforcing kneaded material), considering the passability through the puncture needle, and the ease of solidification in living tissue and the like, the volume of the mixing solution per gram of the substance containing a calcium phosphate-based bone reinforcing material is preferably within a range from about 0.3 mL/g to 0.5 mL/g, and more preferably within a range from about 0.35 mL/g to 0.5 mL/g.

By using a pure gold particle-containing calcium phosphate-based bone reinforcing kneaded material, prepared by kneading together pure gold particles and a substance containing a calcium phosphate-based bone reinforcing material, as a lesion identification marker, the kneaded material is in a paste form and can be injected by a puncture needle before placement, whereas after injection into the body, the lesion identification marker containing the pure gold particles and a substance containing a calcium phosphate-based bone reinforcing material can be retained inside the tissue or the like in the body as a spherical solid or the like.

In those cases where a pure gold particle-containing calcium phosphate-based bone reinforcing kneaded material, prepared by kneading together pure gold particles and a substance containing a calcium phosphate-based bone reinforcing material, is used as a lesion identification marker, a kneaded material containing a high concentration of pure gold particles such that the weight ratio between the pure gold particles and the substance containing a calcium phosphate-based bone reinforcing material (powder) is at least about 1:2 (pure gold particle concentration: about 30 wt %) is preferred, and a kneaded material containing pure gold particles in a concentration that yields a weight ratio between the pure gold particles and the substance containing a calcium phosphate-based bone reinforcing material within a range from 1:2 (pure gold particle concentration: 33 wt %) to 2:1 (pure gold concentration: 66 wt %) is more preferred.

With a pure gold particle-containing calcium phosphate-based bone reinforcing kneaded material, which is a mixture obtained by kneading together pure gold particles and a substance containing a calcium phosphate-based bone reinforcing material, there is a possibility that the ease of discharge from a thin injection needle and the level of leakage of the paste from the placement site in the case of placement in an organ may be related to the viscosity of the paste. The kneaded material obtained from a mixing solution and a substance containing a calcium phosphate-based bone reinforcing material, or the kneaded material obtained from pure gold particles, a mixing solution, and a substance containing a calcium phosphate-based bone reinforcing material, preferably has a viscosity at 20° C. of 10⁸ to 10¹⁰ mPa·s about 5 min after the start of kneading.

### <Lesion Identification Marker Kit for Use in Radiation Therapy>

A lesion identification marker kit for use in radiation therapy according to an embodiment of the present invention includes the pure gold particles described above and the substance containing a calcium phosphate-based bone reinforcing material described above. The lesion identification marker kit for use in radiation therapy according to an embodiment of the present invention includes pure gold particles, and a substance containing a calcium phosphate-based bone reinforcing material for which the volume mean diameter (MV) is within a range from 3 to 12 μm. The lesion identification marker kit for use in radiation therapy according to an embodiment of the present invention preferably includes a substance containing a calcium phosphate-based bone reinforcing material for which the volume mean diameter (MV) is within a range from 3 to 12 μm, and pure gold particles having a median diameter (D50, volume basis) of 16 to 40 μm, and more preferably includes a substance containing a calcium phosphate-based bone reinforcing material for which the volume mean diameter (MV) is within a range from 3 to 12 μm, and pure gold particles having a median diameter (D50, volume basis) of 20 to 35 μm. The lesion identification marker kit for use in radiation therapy according to the present embodiment may, if necessary, include the mixing solutions described above for preparing a kneaded material of a substance containing a calcium phosphate-based bone reinforcing material, or for preparing a pure gold particle-containing calcium phosphate-based bone reinforcing kneaded material by kneading together pure gold particles and a substance containing a calcium phosphate-based bone reinforcing material.

When pure gold particles representing a lesion identification marker are to be placed in any site in the body, a calcium phosphate-based bone reinforcing kneaded material may be prepared by mixing the mixing solution and the substance containing a calcium phosphate-based bone reinforcing material included in the kit. Alternatively, when a kneaded material of pure gold particles and a substance containing a calcium phosphate-based bone reinforcing material (pure gold particle-containing calcium phosphate-based bone reinforcing kneaded material) that represents a lesion identification marker is to be placed in any site in the body, a pure gold particle-containing calcium phosphate-based bone reinforcing kneaded material may be prepared by mixing the pure gold particles, the mixing solution, and the substance containing a calcium phosphate-based bone reinforcing material included in the kit. Alternatively, a pure gold particle-containing calcium phosphate-based bone reinforcing kneaded material may also be prepared by mixing a mixture of pure gold particles and a substance containing a calcium phosphate-based bone reinforcing material included in the kit with a mixing solution included in the kit immediately before being placed in any site in the body.

The lesion identification marker kit for use in radiation therapy according to an embodiment of the present invention may further include a kneading tool, an injection syringe, a puncture needle, a catheter, a wire, or an injection device or the like.

### EXAMPLES

Herein, the present invention is described in more detail with reference to examples and comparative examples, but the present invention is in no way limited to the following examples.

In the present examples, a substance containing a calcium phosphate-based bone reinforcing material is sometimes referred to as a CPC, pure gold particles as G, and further, a mixture of pure gold particles and a substance containing a calcium phosphate-based bone reinforcing material, and the kneaded materials, markers, and marker clusters generated therefrom are respectively referred to as G/CPC mixtures, G/CPC pastes, G/CPC markers, and G/CPC marker clusters.

### Example 1

[Pure Gold Concentration (Wt %) in G/CPC Mixture and Image Recognition Performance (1)]

The image recognition performance of G/CPC mixtures using pure gold microparticles as X-ray markers were investigated for G/CPC mixtures having pure gold particle weight concentrations of [1] 0 wt % to, [2] 20 wt %, [3] 33 wt %, and [4] 50 wt %.

(1) Experimental Method

1) Preparation of G/CPC Kneaded Materials

About 3 g of Biopex-R (Long Type) (HOYA Technosurgical Corporation; medical device approval number: 21300BZZ00274000), which is a substance containing a calcium phosphate-based bone reinforcing material (CPC), various weights of powdered pure gold particles (The Nilaco Corporation; particle size: 1 to 2 μm; purity: 99.99 wt % (product number: AU-174015)), and about 1 mL of a dedicated mixing solution for Biopex-R were mixed in the supplied mortar to produce the calcium phosphate-based bone reinforcing kneaded materials in paste form. The prepared kneaded materials, as shown in Table 1, represent four types of kneaded materials having different ratios between the weight of pure gold particles (g) and the weight of the Biopex-R powder (g) used (weight of pure gold particles (g): [1] weight of Biopex-R powder (g)=0:1 (pure gold particle concentration: 0 wt %); [2] 1:4 (pure gold particle concentration: 20 wt %); [3] 1:2 (pure gold particle concentration: 33 wt %); or [4] 1:1 (pure gold particle concentration: 50 wt %)). Approximately constant volumes (about 10, 30, or 100 μL) of these were injected into a 96-well microplate (round bottom) with n=3 to 4 using a micropipette. In order to calculate the weight of pure gold particles per injected sample of the G/CPC kneaded materials, the injected weight (mg) was measured for each and every sample in which gold particles were kneaded into the paste, and the weight of pure gold particles (mg) was calculated for the injection locations (see Table 2). The weight of the dedicated mixing solution (g) used in the preparation of the kneaded material above was calculated by multiplying the liquid amount used (mL) by the specific gravity of the liquid (1.1085) determined at the time of the present test. The samples prepared in the manner above were provided for image recognition performance testing as described below.

TABLE 1
Preparative conditions for various paste form pure gold particle/CPC kneaded materials

| Test | Target | | | | | | | | Times recommended |
|---|---|---|---|---|---|---|---|---|---|
| kneaded | concentration | | Weight of | | | | Liquid amount | | liquid amount (mL) |
| material | of pure gold | | pure gold | | Weight of | | of dedicated | | of dedicated |
| number | particles | | particles | | CPC powder | | mixing solution | | mixing solution (*1) |
| P01 | 0 | wt % | 0 | g | 3.0 | g | 1.0 | mL | 1.25× |
| P02 | 20 | wt % | 0.725 | g | 2.94 | g | 0.98 | mL | 1.25× |
| P03 | 33 | wt % | 1.468 | g | 2.92 | g | 0.97 | mL | 1.25× |
| P04 | 50 | wt % | 2.890 | g | 2.911 | g | 1.152 | mL | 1.48× |

(*1: calculated using a recommended liquid amount of 1.6 mL of the dedicated mixing solution per 6 g of the CPC powder)

TABLE 2
Pure gold particle content (mg) in each injected sample when various
kneaded materials were injected into 96-well plate

| | Pure | | | | | | | | | | | | |
|---|---|---|---|---|---|---|---|---|---|---|---|---|---|
| | gold | | | | | | | | | | | | |
| Test | particle | | | | | | | | | | | | |
| kneaded | target | | | | | | | | | | | | |
| material | concen- | Injection amount | | | | | | | | | | | |
| number | tration | 10 μL | | | | 30 μL | | | | 100 μL | | | |
| P01 | 0 wt % | 0 | 0 | 0 | — | 0 | 0 | 0 | — | 0 | 0 | 0 | 0 |
| P02 | 20 wt % | 3.2 | 2.5 | 3.4 | — | 9.3 | 7.3 | — | 8.5 | — | 24 | 22 | 31 |
| P03 | 33 wt % | 5.5 | 5.2 | 5.7 | — | 19 | 19 | 17 | 21 | — | 68 | 71 | 65 |
| P04 | 50 wt % | 5.4 | 6.0 | 9.1 | 7.3 | 33 | 37 | 33 | 36 | 60 | 92 | 61 | 74 |

(values in the table represent the pure gold particle content (mg) in the injected sample of each paste form kneaded material)

It has been reported (J. Soc. Inorganic Mat. Jap., Vol. 12, p. 262 (2005)) that the powder composition of Biopex-R includes α-tricalcium phosphate (75 wt %), tetracalcium phosphate (18 wt %), calcium hydrogen phosphate (5 wt %), hydroxyapatite (2 wt %), and magnesium phosphate, and further, that the dedicated mixing solution includes disodium succinate anhydride (12 wt %), sodium chondroitin sulfate ester (5 wt %), sodium bisulfite, and water for injection (83 wt %).

2) Evaluation of Visibility and Image Recognition Performance

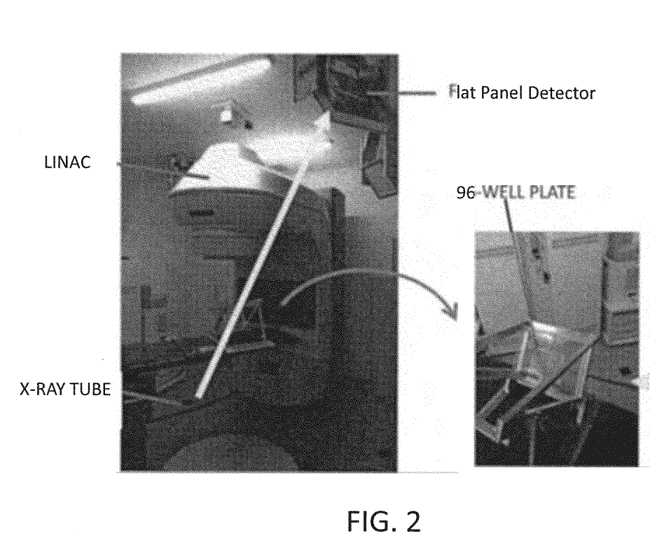

The 96-well plate into which the pure gold particle/CPC mixtures from above had been injected and then solidified was placed on an acrylic phantom, and an X-ray fluoroscopy device (X-ray generation device: UD150B-40 manufactured by Shimadzu Corporation; flat panel detector for X-ray image acquisition (see FIG. 2): PaxScan 3030 manufactured by Varian Medical Systems, Inc.) was used to acquire X-ray fluoroscopy images (the linac in FIG. 2 was not used in the test). The weight and concentration of pure gold particles in the samples filling the wells of the 96-well plate are shown in Table 2. The 1.5 mm and 2.0 mm diameter pure gold spherical markers (iGold) that are currently used clinically were placed as positive controls. The acrylic plate thickness was gradually changed from 1 cm to 25 cm, the tube voltage of the X-ray generation device was fixed at 110 kV, the exposure time was fixed at 3 msec, the tube current was selected from 50 mA, 80 mA and 160 mA depending on the situation, and about 100 X-ray fluoroscopy images were acquired under each of the conditions. The marker visibility (ease of viewing the marker in the image) was objectively (qualitatively) evaluated from the X-ray fluoroscopy images of the 96-well plate containing the pure gold particle markers obtained under each of the conditions.

Furthermore, a template image was created by cutting out an image of the pure gold particle marker targeted for evaluation from one of the plurality of images under each of the conditions, and the image recognition performance was then evaluated with respect to the other images by performing template pattern matching by normalized cross-correlation with the template image created beforehand. When the average value of a correlation coefficient obtained from template pattern matching for about 100 images exceeded 0.3, image recognition was determined to be possible (∘), while image recognition was deemed not possible (x) for lower values. An image processing library (Matrox Imaging Library 9 manufactured by Matrox Corporation) was used for image gradation processing and pattern matching. Table 3 shows the results only for a representative case where the pure gold particle content was 33 wt %.

(2) Results

When the mixing ratio by weight of pure gold particles (particle size: 1 to 2 μm) and the CPC was at least 1:2 (pure gold concentration: 33 wt %) and the injection amount of the paste was 30 to 100 μL, visibility equivalent to, or higher than, the 2 mm diameter or 1.5 mm diameter pure gold spherical markers of the positive controls was observed. Furthermore, the trend was the same under the other evaluation conditions (acrylic thickness, X-ray tube current). In other words, when the amount of pure gold particles was at least about 20 mg, visibility equivalent to, or higher than, the 2 mm diameter or 1.5 mm diameter pure gold spherical markers of the positive controls was observed. Moreover, in the evaluation by template pattern matching, when the amount of pure gold particles was at least about 20 mg, image recognition performance equivalent to or higher than the 2 mm diameter or 1.5 mm diameter pure gold spherical markers of the positive controls was achieved (see Table 3).

Furthermore, the kneaded material that includes a pure gold particle content of about 5 mg (test kneaded material number: P03) was shown to have favorable image recognition performance when the X-ray tube current was either 50 mA or 80 mA, and provided the acrylic plate thickness was 15 cm or less. Moreover, although the details of the data are not particularly specified, it was evident that the cases of test kneaded material numbers P02 and P04 similarly had favorable X-ray image recognition performance when the pure gold particle content was at least about 5 mg but less than 20 mg, and provided the acrylic plate thickness was 15 cm or less.

TABLE 3
Image recognition performance evaluation of gold particle/CPC
mixtures having a pure gold particle content of 33 wt %

| | Acrylic plate | Pure gold content of marker in image | | | | | | | | iGold | |
|---|---|---|---|---|---|---|---|---|---|---|---|
| Condi- | thickness/ | recognition performance evaluation | | | | | | | | (spherical) | |
| tion | X-ray tube | 5.5 | 5.2 | 5.7 | 19 | 17 | 68 | 71 | 65 | 1.5 | 2.0 |
| No. | current | mg | mg | mg | mg | mg | mg | mg | mg | mm | mm |
| 1 | 1cm/50mA | o | o | o | o | o | o | o | o | o | o |
| 2 | 5cm/50mA | o | o | o | o | o | o | o | o | o | o |
| 3 | 10cm/50mA | o | o | o | o | o | o | o | o | o | o |
| 4 | 10cm/80mA | o | o | o | o | o | o | o | o | o | o |
| 5 | 15cm/50mA | o | o | o | o | o | o | o | o | o | o |
| 6 | 15cm/80mA | o | o | o | o | o | o | o | o | o | o |
| 7 | 20cm/50mA | x | x | x | x | x | o | o | o | o | o |
| 8 | 20cm/80mA | x | x | x | o | o | o | o | o | o | o |
| 9 | 25cm/50mA | x | x | x | x | x | x | x | x | x | x |
| 10 | 25cm/80mA | x | x | x | x | x | x | x | x | x | x |
| 11 | 25cm/160mA | x | x | x | x | x | o | o | x | x | x |

(Note: o: image recognition possible; x: image recognition not possible)

### Example 2

[Verification of Tracking Performance by Image Recognition of G/CPC Mixtures]

(1) Experimental Method

The 96-well plate containing the G/CPC mixtures prepared in Example 1 was placed on a movable table capable of one-dimensional drive control, which was operated on a chest phantom (LUNGMAN manufactured by Kyoto Kagaku Co., Ltd.) to simulate a breathing motion, and an X-ray fluoroscopy device (X-ray generation device: UD150B-40 manufactured by Shimadzu Corporation; flat panel detector for X-ray image acquisition: PaxScan 3030 manufactured by Varian Medical Systems, Inc.) was used to acquire X-ray fluoroscopy images. This enabled the situation of a pure gold particle marker that moves within the lungs due to breathing and the like to be reproduced. The tube voltage of the X-ray generation device was 110 kV, the exposure time was 3 msec, the imaging frequency was 15 times/sec, the tube current was 80 mA, and about 400 X-ray fluoroscopy images were acquired. A template image was created by cutting out an image of the pure gold particle marker targeted for evaluation from one of the plurality of images, and whether the pure gold particle marker moving in the images can be tracked by image recognition (tracking performance) was evaluated with respect to the series of about 400 acquired images by performing template pattern matching by normalized cross-correlation with the template image created beforehand. An image processing library (Matrox Imaging Library 9 manufactured by Matrox Corporation) was used for image gradation processing and pattern matching.

(2) Results

Even in a heterogeneous image such as that of X-ray fluoroscopy of an actual human body, when the mixing ratio by weight of pure gold particles (particle size: 1 to 2 μm) and the CPC was at least 1:2 (pure gold concentration: 33 wt %) and the injection amount of the paste was 30 to 100 μL, visibility equivalent to, or higher than, the 2 mm diameter or 1.5 mm diameter pure gold spherical markers of the positive controls was observed. Furthermore, the results of dynamic verification of the tracking performance indicated that pure gold particle markers containing at least about 20 mg of pure gold have sufficient tracking performance to enable tracking that follows movements in the same manner as the 2 mm diameter or 1.5 mm diameter spherical pure gold spherical markers of the positive controls.

### Example 3

[Pure Gold Concentration (Wt %) in G/CPC Mixtures and Image Recognition Performance (2)]

An investigation was conducted to ascertain whether or not there was a significant difference in the image recognition performance between pure gold particle concentrations of 30 wt %, 33 wt %, and 40 wt %. Furthermore, using the same method as Example 1, an investigation was conducted as to whether or not there was a significant difference in the image recognition performance resulting from a difference in the particle width of the pure gold particles.

(1) Experimental Method

1) Preparation of G/CPC Kneaded Materials

Three types of kneaded materials having different pure gold particle content ratios (pure gold particle concentration: 30 wt %, 33 wt %, or 40 wt %) were prepared. Approximately constant volumes (about 30 or 100 μL) of these materials were injected into a 96-well microplate (round bottom) with n=3 using a micropipette, and the pure gold particle content (mg) for the injection locations was also calculated in the same manner as Example 1 (see Table 4). At the time of preparing the G/CPC kneaded materials, about 0.4 mL of the dedicated mixing solution was used per gram of the CPC.

TABLE 4
Gold content (mg) in each injected sample for pure gold/CPC
kneaded materials having gold particle concentrations
of 30 wt %, 33 wt %, and 40 wt %

| Test kneaded | Target | | | | | | |
|---|---|---|---|---|---|---|---|
| material | concentration | Injection amount (μL) | | | | | |
| number | of gold particles | 30 μL | | | 100 μL | | |
| P05 | 30 wt % | 16.2 | 17.4 | 17.4 | 54.0 | 53.1 | 60.0 |
| P06 | 33 wt % | 19.3 | 18.3 | 19.3 | 64.5 | 54.7 | 62.2 |
| P07 | 40 wt % | 26.6 | 26.8 | 25.6 | 84.5 | 88.3 | 87.1 |

(values in the table represent the gold particle content (mg) in the injected sample of each paste form kneaded material)

2) Evaluation of Image Recognition Performance

The 96-well plate containing the pure gold particle/CPC mixtures from above was placed on an acrylic phantom, and an X-ray fluoroscopy device (X-ray generation device: UD150B-40 manufactured by Shimadzu Corporation; flat panel detector for X-ray image acquisition: PaxScan 3030 manufactured by Varian Medical Systems, Inc.) was used to acquire X-ray fluoroscopy images. The 1.5 mm and 2.0 mm diameter pure gold spherical markers (iGold) that are currently used clinically were placed as positive controls. The acrylic plate thickness was gradually changed from 1 cm to 25 cm, the tube voltage of the X-ray generation device was fixed at 110 kV, the exposure time was fixed at 3 msec, the tube current was selected from 50 mA, 80 mA and 160 mA depending on the situation, and about 100 X-ray fluoroscopy images were acquired under each of the conditions. A template image was created by cutting out an image of the pure gold particle marker targeted for evaluation from one of the plurality of images under each of the conditions, template pattern matching was performed with respect to the other images by normalized cross-correlation with the template image created beforehand, and when the average value of a correlation coefficient obtained from template pattern matching for about 100 images exceeded 0.3, image recognition was determined to be possible (∘), while image recognition was deemed not possible (x) for lower values. An image processing library (Matrox Imaging Library 9 manufactured by Matrox Corporation) was used for image gradation processing and pattern matching.

(2) Results

Although detailed data is not recorded, the results of evaluating whether or not image recognition was possible with respect to mixtures using pure gold particles manufactured by The Nilaco Corporation (particle size: 1 to 2 μm) having pure gold concentrations of 30 wt %, 33 wt %, and 40 wt % indicated that the image recognition performance correlated with the pure gold content of the G/CPC markers in each of the cases, with no significant difference observed between pure gold concentrations of 30 to 40 wt %, and G/CPC markers having a pure gold content of at least about 20 mg displaying image recognition performance equivalent to pure gold spherical markers.

### Example 4

[Pure Gold Concentration (Wt %) in G/CPC Mixtures and Image Recognition Performance (3)]

The image recognition performance when the pure gold particle weight concentration in the G/CPC mixture was 66 wt % and 80 wt % was investigated using the same method as Example 1.

(1) Experimental Method

1) Preparation of G/CPC Kneaded Material

In substantially the same manner as Example 1, pure gold particles having a particle width of 33 to 53 μm were mixed with a Biopex-R (Long Type) powder such that the weight concentration in the G/CPC mixture became 33 wt % (pure gold particle weight (g):CPC weight (g)=1:2), 66 wt % (pure gold particle weight (g): CPC weight (g)=2:1), and 80 wt % (pure gold particle weight (g): CPC weight (g)=4:1) and 0.4 to 0.5 mL of the dedicated mixing solution per gram of the CPC was added to each mixture to prepare the G/CPC pastes. In order to form G/CPC marker clusters of various sizes, various volumes of the G/CPC paste were injected into Konnyakubatake (manufactured by Mannanlife Co., Ltd.; a gel-like food product that contains a large amount of dietary fiber consisting of konjac flour, and gelling agents (thickening polysaccharides) and the like), and these samples were left overnight at 37° C. Then, the solidified clusters were extracted and left for 1 to 3 days in a large amount of purified water. The weight of the obtained wet clusters was measured to calculate the gold content included in the G/CPC marker clusters. Small clusters containing 66 wt % and 80 wt % of pure gold particles were prepared by the grinding of large clusters, and the weight of the obtained small clusters was measured to calculate the pure gold particle content. These small clusters were each placed in a 96-well plate (see Table 5), and were used as samples for evaluating the image recognition performance.

TABLE 5
Pure gold content (mg) in G/CPC clusters containing 33 wt %,
66 wt %, and 80 wt % of gold particles

| Gold particles | Pure gold content (mg) in G/CPC clusters | | | | | | | | |
|---|---|---|---|---|---|---|---|---|---|
| in wt% | 33 wt % | | | | 66 wt % | | 80 wt % | | |
| | 70 | 92 | 73 | 65 | 181 | | | 241 | 219 |
| (19) | 36 | 31 | 31 | 39 | 96 | 96 | 74 | 86 | 82 |
| | | | | | (17) | (18) | | | |
| (9) | | 13 | 12 | 10 | 32 | 34 | 34 | 33 | 31 |
| (1) | | 8.1 | 7.5 | 8.6 | 15 | 14 | 20 | 20 | 18 |

The values enclosed in parentheses ((1), (9), and (19)) represent the sample numbers at the left end of the plate.
(17) and (18) are placement locations of the 1.5 mm and 2.0 mm spherical gold markers used as a control.

2) Evaluation of Visibility and Image Recognition Performance

Using the same method as Example 1, the 96-well plate in which the G/CPC marker clusters from above had been deposited was placed on an acrylic phantom, and an X-ray fluoroscopy device (X-ray generation device: UD150B-40 manufactured by Shimadzu Corporation; flat panel detector for X-ray image acquisition: PaxScan 3030 manufactured by Varian Medical Systems. Inc.) was used to acquire and evaluate X-ray fluoroscopy images. The 1.5 mm and 2.0 mm diameter pure gold spherical markers (iGold) that are currently used clinically were placed as positive controls. The acrylic plate thickness was gradually changed from 1 cm to 25 cm, the tube voltage of the X-ray generation device was fixed at 110 kV, the exposure time was fixed at 3 msec, the tube current was selected from 50 mA, 80 mA and 160 mA depending on the situation, and about 100 X-ray fluoroscopy images were acquired under each of the conditions. The marker visibility was objectively evaluated from the X-ray fluoroscopy images of the 96-well plate containing the pure gold particle markers obtained under each of the conditions.

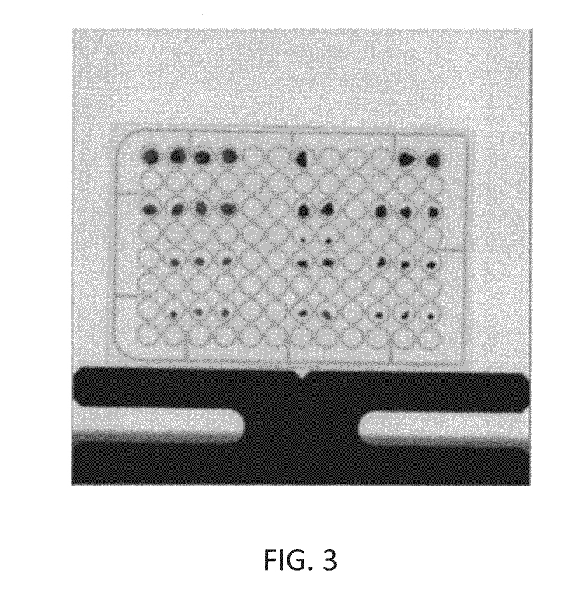

Furthermore, a template image was created by cutting out an image of the pure gold particle marker targeted for evaluation from one of the plurality of images under each of the conditions, template pattern matching was performed with respect to the other images by normalized cross-correlation with the template image created beforehand, and when the average value of a correlation coefficient obtained from the template pattern matching for about 100 images exceeded 0.3, image recognition was determined to be possible (∘), while image recognition was deemed not possible (x) for lower values. An image processing library (Matrox Imaging Library 9 manufactured by Matrox Corporation) was used for image gradation processing and pattern matching. FIG. 3 shows an X-ray fluoroscopy image for an acrylic plate thickness of 1 cm, and a tube current of 50 mA.

(2) Results

Table 6 shows data relating to the ease of image recognition of the G/CPC marker clusters having a pure gold content of 66 wt % and 80 wt %. An improvement in the image recognition performance dependent on the pure gold content was observed in G/CPC marker clusters of both pure gold concentrations, and G/CPC marker clusters that included about 20 mg of pure gold displayed an equivalent image recognition performance to 1.5 mm diameter spherical gold markers.

However, under these kneading conditions, a significant increase in viscosity was observed for the 80 wt % G/CPC paste, making passage through even an 18 to 21 G thin diameter needle impossible, suggesting a need to reexamine the mixing ratio by including the kneading water.

TABLE 6
Image recognition performance of G/CPC clusters containing 66 wt % or 80 wt % of gold clusters

| | Sample No. | | | | | | | | | | | | | | | | | | | |
|---|---|---|---|---|---|---|---|---|---|---|---|---|---|---|---|---|---|---|---|---|
| | 4 | 5 | 12 | 13 | 23 | 24 | 32 | 6 | 7 | 8 | 14 | 15 | 16 | 25 | 26 | 27 | 33 | 34 | 17 | 18 |
| | Pure gold contest (mg) within 66 wt % | | | | | | | Pure gold content (mg) within 80 wt % | | | | | | | | | | | iGold | |
| Pure gold | gold particle sample | | | | | | | gold particle sample | | | | | | | | | | | (mm) | |
| content | 15 | 14 | 32 | 34 | 96 | 96 | 181 | 20 | 20 | 18 | 34 | 33 | 31 | 74 | 86 | 82 | 241 | 219 | 1.5 | 2 |
| 1 cm/50 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 5 cm/50 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 5 cm/80 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 10 cm/50 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 10 cm/80 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 15 cm/50 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 15 cm/80 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 20 cm/50 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 20 cm/80 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 20 cm/160 mA | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ | ∘ |
| 25 cm/80 mA | x | x | x | x | x | x | x | x | x | x | x | x | x | x | x | x | x | x | x | x |
| 25 cm/160 mA | x | x | ∘ | ∘ | ∘ | ∘ | ∘ | x | x | x | ∘ | ∘ | ∘ | ∘ | ∘ | ∘ | ∘ | ∘ | x | ∘ |

### Summary of Examples 1 to 4

The results of Examples 1 to 4 revealed the following.
For G/CPC markers having a pure gold particle concentration of 30 wt % to 80 wt %, the image recognition performance depends on the gold content (mg) included in the G/CPC marker.
For G/CPC markers that include 30 to 80 wt % of pure gold particles (ratio of weight of G:weight of CPC of at least about 1:2 but not more than 4:1), a pure gold content of at least about 20 mg and preferably near-spherical clusters are considered to provide image recognition performance, at least in currently used X-ray fluoroscopy detection devices (flat panel detectors), that is equivalent to, or better than, 1.5 mm or 2.0 mm diameter spherical gold markers that are currently commercially available as X-ray markers.

### Example 5

[Evaluation of Image Recognition Performance of G/CPC Kneaded Materials Having Different Pure Gold Particle Widths]

Using the same method as Example 1, an investigation was conducted as to whether or not there was a significant difference in the image recognition performance due to differences in the particle width of the pure gold particles.

(1) Experimental Method

1) Preparation of G/CPC Kneaded Materials

About 2 g of Biopex-R (Long Type) (manufactured by HOYA Technosurgical Corporation), about 1 g of pure gold particles having one of four different particle widths (particle width of 32 μm or less, 33 to 53 μm, 54 to 75 μm, and 76 to 150 μm), and 0.8 mL of a dedicated mixing solution for Biopex-R were mixed using the supplied mortar to prepare four types of kneaded materials in paste form as shown in Table 7. Approximately constant volumes (about 30 or 100 μL) of these were injected into a 96-well microplate (round bottom) with n=3, and the pure gold particle content in each injection location was also calculated in the same manner as Example 4 (see Table 8). The G/CPC pastes injected into the 96-well microplate had solidified several hours later, and these were provided for testing as described below. Furthermore, the pure gold particles having particle widths of 32 μm or less, 33 to 53 μm, 54 to 75 μm, and 76 to 150 μm that were used each had a purity of at least 99.99 wt %, and were pure gold microparticles obtained by spraying pure gold that had been heated and melted using an atomizer, followed by fractionation using a sieve.

TABLE 7
Preparation of pure gold particle/CPC kneaded materials
with pure gold particles having different particle widths

| Test | Target | | | Liquid amount | Times recommended |
|---|---|---|---|---|---|
| kneaded | concentration | Weight of | | of dedicated | liquid amount (mL) |
| material | of gold | gold particles | Weight of | mixing solution | of dedicated |
| number | particles | (particle width) | CPC powder | (volume) | mixing solution (*1) |
| P08 | 33 wt % | 1.002 g | 2.004 g | (0.8 mL) | 1.50× |
| | | (32 μm or less) | | 0.887 g | |
| P09 | 33 wt % | 1.019 g | 2.005 g | (0.8 mL) | 1.50× |
| | | (33-53 μm) | | 0.887 g | |
| P10 | 33 wt % | 1.083 g | 2.060 g | (0.8 mL) | l.46× |
| | | (54-75 μm) | | 0.887 g | |
| P11 | 33 wt % | 1.011 g | 2.017 g | (0.8 mL) | 1.49× |
| | | (76-150 μm) | | 0.887 g | |

(*1: calculated using a recommended volume of 1.6 m/L of the dedicated mixing solution per 6 g of the CPC powder)

TABLE 8
Weight of gold (mg) in each injected sample of gold particle/CPC
kneaded materials having different gold particle widths

| Test | Target | | | | | | |
|---|---|---|---|---|---|---|---|
| kneaded | concentration of | | | | | | |
| material | gold particles | Injection amount (μL) | | | | | |
| number | (wt %) | 30 μL | | | 100 μL | | |
| P08 | 33 wt % | 18.3 | 16.3 | 18.9 | 63.3 | 64.3 | 63.3 |
| P09 | 33 wt % | 19.5 | 19.3 | 19.3 | 57.1 | 64.9 | 65.1 |
| P10 | 33 wt % | 18.6 | 21.2 | 19.1 | 65.2 | 62.9 | 60.7 |
| P11 | 33 wt % | 19.0 | 15.4 | 20.6 | 67.9 | 65.1 | 63.3 |

(values in the table represent the gold particle content (mg) in the injected sample of each paste form kneaded material)

2) Evaluation of Image Recognition Performance

In the same manner as Example 3, the 96-well plate containing the G/CPC mixtures from above was placed on an acrylic phantom, and an X-ray fluoroscopy device (X-ray generation device: UD150B-40 manufactured by Shimadzu Corporation; flat panel detector for X-ray image acquisition: PaxScan 3030 manufactured by Varian Medical Systems, Inc.) was used to acquire and evaluate X-ray fluoroscopy images.

(2) Results

Although Table 9 shows the results of image recognition evaluation for only pure gold particles having a particle width of 32 μm or less, the results of evaluating whether or not image recognition was possible with mixtures of pure gold particles respectively having particle widths of 32 μm or less, 33 to 53 μm, 54 to 75 μm, and 76 to 150 μm revealed no significant difference arising from the particle width of the pure gold particles (between 32 μm or less and 150 to 75 μm). Furthermore, in each of the pure gold particles manufactured by The Nilaco Corporation (particle size: 1 to 2 μm), image recognition performance equivalent to, or higher than, the 2 mm diameter or 1.5 mm diameter pure gold spherical markers of the positive controls was observed, and it is thought that there is essentially no significant difference in the image recognition performance arising from the particle width of the pure gold particles.

TABLE 9
Image recognition evaluation using a gold particle width of 32 microns or less

| | | Pure gold content (mg) of marker in | | | | | | |
|---|---|---|---|---|---|---|---|---|
| Condition | Acrylic | degree of recognition evaluation | | | | | | Spherical |
| No. | thickness/current | 18.3 | 16.3 | 18.9 | 63.3 | 64.3 | 63.3 | marker |
| 1 | 1 cm/50 mA | ∘ | ∘ | ∘ | ∘ | ∘ | ∘ | ∘ |
| 2 | 5 cm/50 mA | ∘ | ∘ | ∘ | ∘ | ∘ | ∘ | ∘ |
| 3 | 10 cm/50 mA | ∘ | ∘ | ∘ | ∘ | ∘ | ∘ | ∘ |
| 4 | 10 cm/50 mA | ∘ | ∘ | ∘ | ∘ | ∘ | ∘ | ∘ |
| 5 | 15 cm/50 mA | ∘ | ∘ | ∘ | ∘ | ∘ | ∘ | ∘ |
| 6 | 15 cm/80 mA | ∘ | ∘ | ∘ | ∘ | ∘ | ∘ | ∘ |
| 7 | 20 cm/50 mA | x | ∘ | x | ∘ | ∘ | ∘ | ∘ |
| 8 | 20 cm/80 mA | ∘ | ∘ | ∘ | ∘ | ∘ | ∘ | ∘ |
| 9 | 25 cm/50 mA | x | x | x | x | x | x | x |
| 10 | 25 cm/80 mA | x | x | x | x | x | ∘ | x |
| 11 | 25 cm/160 mA | x | x | x | x | x | ∘ | x |

### Example 6

[G/CPC Marker Placement Test Using PBS-Perfused Pig Liver]

In G/CPC paste placement testing using a pig liver preserved by freezing or preserved by refrigeration, the G/CPC marker clusters extracted from the liver did not form large clusters, and in many cases exhibited a particle-like form. Therefore, the existence or absence of marker cluster formation was confirmed in a pig liver perfused with a simulated body fluid at about 37° C.

(1) Experimental Method

2 g of K₂HPO₄, 2 g of KCl, 11.5 g of Na₂HPO₄, and 80 g of NaCl were dissolved while being stirred in pure water to prepare 1000 mL of simulated body fluid (Dulbecco's Phosphate-Buffered Saline(−), referred to as D-PBS(−) below). The D-PBS(−) was diluted by 10 times with pure water, and heated to at least 37° C. in a constant temperature bath. A silicon tube was ligated to the stem hepatic vein or portal vein of the pig liver. The liver was placed on a net, the silicon tube was connected to a peristaltic pump and perfused with the heated 10 times-diluted buffer (D-PBS(−)) from above. After the surface of the pig liver reached substantially 37° C., the G/CPC pastes with the compositions shown in Table 10 were placed using an 18 G puncture needle with a needle length of 20 cm. After placement of the markers, the heated 10 times-diluted D-PBS(−) was passed through the interior of the liver for about 1 hour, maintaining the temperature at about 37° C. X-ray fluoroscopy imaging was carried out by placing 1.5 mm and 2 mm pure gold markers in the liver, and the marker clusters were extracted thereafter from the liver.

TABLE 10
Preparation of kneaded material containing
pure gold particles and a CPC

| Target | | | |
|---|---|---|---|
| concentration | Weight of pure | Weight of | Liquid amount |
| of pure gold | gold particles | CPC powder | of dedicated |
| particles | (particles width) | (product type) | mixing solution |
| 33 wt % | 2.01 g | 4.04 g | 1.6 mL |
| | (33-53 μm) | (Biopex-R Long | |
| | | Type) | |

In the X-ray fluoroscopy, the pig liver was placed on a human X-ray phantom, and an X-ray fluoroscopy device (Artis Zee Celling manufactured by Siemens AG) was used to perform X-ray fluoroscopy. Further, a computer tomographic image was taken using a computer tomographic imaging device (SOMATOM Definition AS64 manufactured by Siemens AG) to measure the X-ray absorption value (CT value), and the visibility was evaluated.

(2) Results

The results are shown in Table 11.

TABLE 11

| No | 1 | 2 | 3 | 4 | 5 | 6 |
|---|---|---|---|---|---|---|
| Marker shape | small | spherical | spherical | spherical | ellipsoid | cylindrical |
| | fragment | | | | | |
| State | | bound to | bound to | bound to | bound to | in blood |
| | | tissue | tissue | tissue | tissue | vessel, not |
| | | | | | | bound |
| Weight | — | 146 mg | 104 mg | 118 mg | 119 mg | 86 mg |
| Gold content | — | 37.6 mg | 26.8 mg | 30.4 mg | 30.7 mg | 22.1 mg |
| (calculated value) | | | | | | |
| X-ray visibility | x | ∘ | ∘ | ∘ | ∘ | Δ |
| CT value | — | 3,068 | 3,071 | 3,003 | 3,069 | 3,062 |

Except for No. 1 and No. 6, marker formation with sufficient visibility was observed in all cases even when compared to iGold with φ 2 mm. About 100 mg of the marker clusters were extracted, and sufficiently hard marker clusters had been formed, such that the) could not be crushed even when a force of at least 1 kg was applied. However, it is thought that No. 6 was injected into a blood vessel, and the cylindrical shape led to reduced visibility compared to the other clusters. Furthermore, the clusters of No. 2 to No. 5 were strongly embedded in the tissue.

Although the data is not specifically shown, in a similar G/CPC marker placement test using a PBS-perfused pig liver, there were many cases where small, particle-like clusters were formed due to differences in the G/CPC paste placement site, or the water content of the paste or the like, suggesting that water at the placement site can affect the shape of the cluster and the formation of high-visibility clusters. Furthermore, in a case where Cerapaste (NGK Spark Plug Co., Ltd.), which is an equivalent CPC to Biopex, was kneaded using the same mixing ratio as the test kneaded material No. P22 in Example 7, hard G/CPC marker clusters were formed in the same manner as the present test even when kneading was performed using a mixing ratio in which the amount of water for injection used was 0.1 mL less than in P22 and the like. Moreover, when the G/CPC paste was placed in a pig liver, G/CPC markers containing at least about 5 mg of gold particles as a pure gold content allowed visibility sufficient for observation by X-ray fluoroscopy on a human pelvic phantom.

### Example 7

[Passability of Pure Gold Particles Having Different Particle Widths Through Thin Injection Needle]

Provisionally assuming that the time from the start of kneading to injection to the target site from an injection needle is within about 5 min, the passability of the G/CPC pastes through a thin injection needle at room temperature (about 20° C.) within about 5 min from the start of kneading was investigated using pure gold particles having different particle widths.

(1) Sample Preparation Method

About 2 g of Biopex-R (Long Type) powder and about 1 g of pure gold particles (particle width: 32 μm or less, 33 to 53 μm, 54 to 75 μm, or 76 to 150 μm) were mixed in the supplied mortar, and by mixing the resulting mixture with a dedicated mixing solution for Biopex, or alternatively, by mixing the resulting mixture with about 1 g of Cerapaste powder, about 0.5 g of pure gold particles (particle width: 32 μm or less), or a Cerapaste hardening liquid or the like, five types of pure gold particle/CPC kneaded materials were produced (see Table 12). In terms of the dedicated mixing solution, 0.8 mL of a dedicated mixing solution for Biopex-R was used when Biopex-R was used as the CPC powder, and 0.3 mL of a Cerapaste hardening liquid and 0.2 mL of a viscosity-adjusting water (water for injection) were used when Cerapaste was used as the CPC powder. One mL capacity syringes (manufactured by Medallion Inc.) filled with an appropriate amount of the kneaded materials were fitted with a 22 G metallic needle (needle length: about 4 cm, inner diameter: about 0.5 mm), and an investigation was conducted as to whether or not discharge was possible from the injection needle at room temperature (about 20° C.) within about 5 min after kneading.

TABLE 12
Preparation of kneaded materials containing different CPC product
types and pure gold particles having different particle widths

| | Target | | | |
|---|---|---|---|---|
| Test kneaded | concentration of | Weight of pure gold | | Liquid amount of |
| material | pure gold | particles (particle | Weight of CPC powder | dedicated mixing |
| number | particles | width) | (product type) | solution |
| P18 | 33 wt % | 1.010 g | 2.008 g | 0.8 mL |
| | | (32 μm or less) | (Biopex-R Long Type) | |
| P19 | 33 wt % | 1.040 g | 2.038 g | 0.8 mL |
| | | (33-53 μm) | (Biopex-R Long Type) | |
| P20 | 33 wt % | 1.03 g | 2.05 g | 0.8 mL |
| | | (54-75 μm) | (Biopex-R Long Type) | |
| P21 | 33 wt % | 1.00 g | 2.10 g | 0.8 mL |
| | | (76-150 μm) | (Biopex-R Long Type) | |
| P22 | 33 wt % | 0.508 g | 1.050 g | 0.3 mL (hardening |
| | | (32 μm or less) | (Cerapaste) | liquid) + 0.2 mL (water |
| | | | | for injection) |

(2) Results

The five types of paste-like kneaded materials from above all passed through the 22 G injection needle without a problem. However, they were unable to pass through a 25 G injection needle (needle length: about 4 cm, inner diameter: about 0.25 mm). It may be stated that a kneaded material in which Biopex-R Long Type and pure gold particles having a particle width of 150 μm or less are mixed with a ratio of 33 wt % and then made into a paste form using a dedicated mixing solution is capable of passage through a 22 G injection needle.

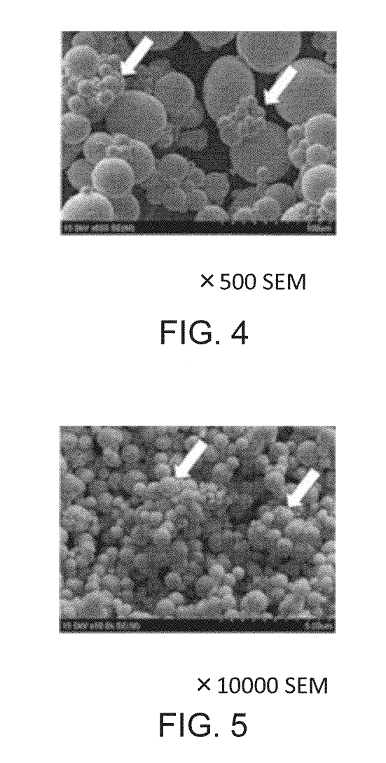

Although the data is not specifically shown, when a kneaded material of gold particles manufactured by The Nilaco Corporation (particle size: 1 to 2 μm, pure product number: AU-174015) and Biopex-R (Long Type) was prepared in the same manner as above, the material was unable to be discharged from a 22 G injection needle. However, when the passability through a 22 G injection needle was investigated for a kneaded material prepared in a similar manner after regrinding the gold particles in an agate mortar, the material passed through without a problem. When the particle size distribution of the mortar-ground product of the product manufactured by The Nilaco Corporation (product number: AU-174015; particle size: 1 to 2 μm) used in the present embodiment was measured using laser diffraction and scattering method, two peaks were observed, showing the presence not only of gold particles having a particle size of about 1 μm, but also that about 50% of the gold particles present were within a range from about 10 to 200 μm. This suggests that, in the case of small pure gold particles manufactured by The Nilaco Corporation, aggregated clusters are easily generated. FIG. 4 and FIG. 5 respectively show the scanning electron microscope (SEM) photographs of pure gold particles having a particle width of 75 to 54 μm, and the mortar-ground product of the product manufactured by The Nilaco Corporation. Aggregation between small particles is particularly frequent in FIG. 4, and further, aggregation of almost all of the small spherical particles into large, grape-like aggregated clusters is observed with a high frequency in FIG. 5, suggesting that aggregated clusters are easily generated in small pure gold particles.

### Example 8

[Passability of G/CPC Pastes with Gold Particles Having Different Particle Widths Through Puncture Needle (Needle Length: 20 cm)]

In Example 7 above, the passability of G/CPC pastes through a thin needle was evaluated using a thin diameter needle with a needle length of about 4 cm, but considering that medical puncture needles are commonly 18 G to 22 G products having a length of 5 cm to 20 cm, and in particular, because products also exist that are used by being fitted to an endoscope, such as puncture needles having a 19 G to 25 G tip section having a length of about 1 m (Medi-Globe Corporation; SonoTip Pro Control), the passability of the G/CPC pastes was evaluated targeting the passability through a 20 G to 22 G thin diameter needle in the form of a puncture needle with a needle length of 20 cm, which is the most commonly used in clinical practice.

If the gold particles manufactured by The Nilaco Corporation (product number: AU-174015: particle size 1 to 2 μm) are not specifically subjected to a grinding treatment or sieving treatment, and 2 g of Biopex, 1 g of the pure gold particles, and 0.8 mL of a mixing solution are kneaded together, passage through an 18 G (inner diameter: 1.07 mm; cross-sectional area: 0.899 mm²) puncture needle (manufactured by Create Medic Co., Ltd.; needle length: 20 cm) represents the limit, and passage did not occur through a 19 G (inner diameter: 0.73 mm; cross-sectional area: 0.418 mm²) puncture needle (manufactured by Create Medic Co., Ltd.; needle length: 20 cm). Upon investigation of the particle size distribution of the pure gold particles (unground product) manufactured by The Nilaco Corporation, particles having the original particle size of 1 to 2 μm were almost undetectable, and instead, the sample was dominated by gold particles in the vicinity of about 300 to 600 μm (particle size representing D50 value: about 446 μm). Furthermore, a gold particle fraction having a particle width of at least 0.3 mm (D50 value: 182 μm: D90 value: 226 μm; cumulative distribution up to particle size of about 400 μm: 100%) was obtained in the preparative process of the pure gold particles by using a sieve having openings of 0.3 mm, a 33 wt % (weight ratio of gold particles and CPC of 1:2) G/CPC paste was prepared using these pure gold particles, the commercially available product Biopex-R and a dedicated mixing solution, and the passability through the puncture needle (needle length: 20 cm) was investigated. Similarly, in this paste, passage through an 18 G puncture needle represented the limit, and passage through a 19 G puncture needle did not occur. In other words, in the G/CPC paste reported as prior art (Non-Patent Document 11), which uses 0.7±0.1 mm or 0.4±0.1 mm diameter pure gold particles and commercially available Biopex, it is thought that, in a similar manner to the two types of pure gold particles above, passage through an 18 G (inner diameter: 1.07 mm; cross-sectional area: 0.899 mm²) puncture needle (needle length: 20 cm) represents the limit.

Therefore, the passability performance of the G/CPC pastes through a puncture needle with a needle length of 20 cm was investigated at room temperature (20 to 25° C.) using pure gold particle fractions having particle widths of 150 to 76 μm, 75 to 54 μm, 53 to 33 μm, and 32 μm or less, which were obtained from pure gold particles prepared by the atomizer method from a pure gold heated melt having a purity of at least 99.99 wt %, and then sequential sieving using sieves having openings (JIS Z 8801) of 150 μm, 75 μm, 53 μm, and 32 μm, and a fraction of 20 μm or less, in which the fraction of 32 μm or less was further fractionated using a 20 μm sieve.

(1) Experimental Method and Results

G/CPC pastes were prepared under the conditions shown in Table 13 using pure gold particles having different particle widths and the commercially available Biopex-R Standard, and the passability of each of the G/CPC pastes through a puncture needle was investigated using puncture needles (manufactured by Togo Medikit Co., Ltd.) with a needle length of 20 cm and different inner diameters of 20 G (inner diameter: 0.70 mm; cross-sectional area: 0.385 mm²), 21 G (inner diameter: 0.59 mm; cross-sectional area: 0.273 mm²), and 22 G (inner diameter: 0.53 mm; cross-sectional area: 0.221 mm²).

TABLE 13

| | | | Weight of | Liquid | | | |
|---|---|---|---|---|---|---|---|
| Test | Target | | CPC | amount of | Puncture needle passability | | |
| kneaded | concentration | Weight of pure | powder | dedicated | (manufactured by Togo Medikit) | | |
| material | of pure gold | gold particles | (product | mixing | 20G | 21G | 22G |
| number | particles | (particle width) | type) | solution | ×20 cm | ×20 cm | ×20 cm |
| P23 | 33 wt % | 1.0 g | 2.0 g | 0.8 mL | ∘ | x | x |
| | | (<20 μm) | (BP-R | | | | |
| | | | Standard) | | | | |
| P24 | 33 wt % | 1.5 g | 3.0 g | 1.2 mL | ∘ | ∘ | x |
| | | (<32 μm) | (BP-R | | | | |
| | | | Standard) | | | | |
| P25 | 33 wt % | 1.5 g | 3.0 g | 1.2 mL | ∘ | Δ/0 | x |
| | | (33-53 μm) | (BP-R | | | | |
| | | | Standard) | | | | |
| P26 | 33 wt % | 1.5 g | 3.0 g | 1.2 mL | ∘ | x/∘ | x |
| | | (54-75 μm) | (BP-R | | | | |
| | | | Standard) | | | | |
| P27 | 33 wt % | 1.5 g | 3.0 g | 1.2 mL | ∘ | x | x |
| | | (76-150 μm) | (BP-R | | | | |
| | | | Standard) | | | | |

Based on this test, it was evident that the gold particle fractions having a particle width of 150 to 76 μm and 20 μm or less were clearly inferior in terms of the passability, whereas the gold particle fractions having a particle width of 53 to 33 μm, and 32 μm or less were preferred, and in particular, the pure gold particle fraction having a particle width of 32 μm or less was able to pass through a 21 G puncture needle with a needle length of 20 cm, and was therefore particularly preferred. In other words, the pure gold particles preferred from the perspective of passing through a long, thin diameter needle are those having a particle width of 53 to 33 μm, and 32 μm or less, and it may be stated that pure gold particles having a particle width of 32 μm or less, but not including particles having a particle width of 20 μm or less, are more preferable. Investigation of the particle size distribution of the fraction having a particle width of 20 μm or less revealed that while a decrease in the D50 value and the D10 value was not observed, the D90 value markedly increased, and in particular, a large shoulder was observed in the portion of the particle size exceeding about 100 μm, and these aggregated pure gold particles are thought to have influenced the passability.

### Example 9

[Passability of G/CPC Pastes Using Sieved Fractions of CPC Through Puncture Needle (Needle Length: 20 cm)]

(1) The various commercially available CPCs were sieved, and the effect on the passability through a puncture needle (needle length: 20 cm) was investigated for CPC-only pastes and G/CPC pastes.

(1-1) Preparation of CPC Pastes

Various Biopex products and Cerapaste (manufactured by NGK Spark Plug Co., Ltd.) were sequentially sieved using sieves having openings (JIS Z 8801) of 75 μm, 53 μm, and 32 μm to obtain CPCs having the respective particle widths of 75 to 54 μm, 53 to 33 μm, and 32 μm or less. In particular, a dedicated mixing solution or a hardening liquid was added to the CPC powder that passed through the 32 μm sieve in the amounts shown in Table 14 to prepare a paste, and the passability of each of the CPC-only pastes was compared with the commercially available products. In terms of the puncture needles, 20 G (inner diameter: 0.70 mm; cross-sectional area: 0.385 mm²), 21 G (inner diameter: 0.59 mm; cross-sectional area: 0.273 mm²), and 22 G (inner diameter: 0.53 mm; cross-sectional area: 0.221 mm²) puncture needles (manufactured by Togo Medikit Co., Ltd.) with a needle length of 20 cm were used.

TABLE 14

| | | Puncture needle passability | | |
|---|---|---|---|---|
| | Mixing | (Togo Medikit) | | |
| | ratio of | 20 G | 21 G | 22 G |
| | mixing | (0.70 | (0.59 | (0.53 |
| CPC type | solution *1 | mm) | mm) | mm) |
| BP-Excellent | 0.4 mL/g | ∘ | x | x |
| BP-Long | 0.4 mL/g | x/∘ | x | x |
| BP-Standard | 0.4 mL/g | ∘ | ∘/x | x |
| Cerapaste | *2 | ∘ | ∘/x | x |
| Sieved BP-Excellent (<32 μm) | 0.4 mL/g | ∘ | ∘ | ∘ |
| Sieved BP-Long (<32 μm) | 0.4 mL/g | ∘ | ∘ | ∘ |
| Sieved BP-Standard (<32 μm) | 0.4 mL/g | ∘ | ∘ | ∘ |
| Sieved Cerapaste (<32 μm) | *2 | ∘ | ∘ | ∘ |

*1: Amount of mixing solution (mL) added per gram of CPC.
*2: Hardening liquid: 0.3 mL + water for injection: 0.2 mL per gram of Cerapaste

(1-2) Results

The fraction of the commercially available product CPC that passed through the 32 μm sieve had a clearly improved ease of passage through the puncture needle (needle length: 20 cm), and the passability through a 22 G puncture needle was confirmed for all of the CPC-only pastes.

(2) In the example above, with the CPC-only pastes, because the powder of the fraction sieved using a 32 μm sieve had particularly favorable passability through the puncture needle (needle length: 20 cm), powders were mixed in which the pure gold particles and the CPC both had different particle widths, and the passability of the various G/CPC pastes through a puncture needle (needle length: 20 cm) was investigated.

(2-1) Preparation of G/CPC Pastes

Using the various CPC fractions that were fractionated in the above test and pure gold particles having different particle widths, pure gold particles, the CPC, and the mixing solution were kneaded together in the same mixing ratios as Example 8 to prepare a series of pastes. For testing, 20 G (inner diameter: 0.70 mm; cross-sectional area: 0.385 mm²), 21 G (inner diameter: 0.59 mm; cross-sectional area: 0.273 mm²), and 22 G (inner diameter: 0.53 mm; cross-sectional area: 0.221 mm²) puncture needles (manufactured by Togo Medikit Co., Ltd.) with a needle length of 20 cm, and 18 G (inner diameter: 1.07 mm; cross-sectional area: 0.899 mm²), 19 G (inner diameter: 0.73 mm; cross-sectional area: 0.418 mm²), 21 G (inner diameter: 0.54 mm; cross-sectional area: 0.229 mm²), and 22 G (inner diameter: 0.54 mm; cross-sectional area: 0.229 mm²) puncture needles (manufactured by Create Medic Co., Ltd.; needle length: 20 cm) were used.

(2-2) Results

Although a portion of the results are shown in Table 15, overall, the cases where the pure gold particles had a particle width of 53 to 33 μm, or 32 μm or less, and the CPC has a particle width of 53 to 33 μm, or 32 μm or less, were preferred, and in particular, when both the pure gold particle and the CPC had particle widths of 32 μm or less, the material was capable of passing through a 22 G puncture needle. In other words, as mentioned in Example 8 above, although the limit of the prior art disclosed in Non-Patent Document 11 (a G/CPC paste using 0.4±0.1 mm pure gold particles and commercially available Biopex) was passage through an 18 G puncture needle (needle length: 20 cm), the G/CPC paste of the present invention was capable of passage through a 22 G puncture needle (needle length: 20 cm). The cross-sectional areas of an 18 G and 22 G puncture needle differ by a factor of about four, and it was evident that notable effects could be obtained by adjusting the particle size distributions of the pure gold particles and the CPC. Among the Biopex powders sieved with a 32 μm sieve, a powder having a particle width of 32 μm or less derived from the Excellent Type had particularly superior passability through a thin diameter needle, even when kneaded together with pure gold particles having a particle width of 32 μm or less.

Although a large portion of the test described above was carried out using about 0.4 mL to 0.5 mL of the mixing solution (or hardening liquid+water for injection) per gram of the CPC, when the passability of the CPC through a 20 cm puncture needle was investigated, the case where 0.35 mL/g of kneading water was mixed was equivalent to the case of 0.4 mL/g, but a declining trend in the passability was observed below 0.30 mL/g. On the other hand, considering that when the particle width of the pure gold particles mixed with the commercially available CPC was changed, there was no significant difference in the viscosity of the G/CPC paste (see Example 11 of PCT International Publication No. WO2016/137013) and the passability through a puncture needle, that the passability of the pastes was substantially equivalent except in the case where the mixing ratio of the pure gold particles and the CPC was extremely high (80 wt %), and that the paste sufficiently hardened inside a perfused pig liver even when 0.5 mL/g of the mixing solution was used, it may be stated that the mixing ratio by volume of the mixing solution per gram of the CPC is preferably within a range from about 0.3 mL/g to 0.6 mL/g, and more preferably within a range from about 0.35 mL/g to 0.5 mL/g.

TABLE 15

| | Togo Medikit (20 cm needle) | | | Create Medic (20 ens needle) | | | |
|---|---|---|---|---|---|---|---|
| | 20G | 21G | 22G | 18G | 19G | 2IG | 22G |
| | (0.70 mm/ | (0.59 mm/ | (0.53 mm/ | (1.07 mm/ | (0.73 mm/ | (0.51 mm/ | (0.51 mm/ |
| CPC + Gold particles | 0.385 mm²) *1 | 0.273 mm²) *1 | 0.221 mm²) *1 | 0.899 mm²) *1 | 0.418 mm²) *1 | 0.204 mm²) *1 | 0.204 mm²) *1 |
| Sieved BP-R Long | ∘ | Δ | Δ | | | | |
| (33-53 μm) + gold | | | | | | | |
| (54-75 μm) | | | | | | | |
| Sieved BP-R Long | | | ∘ | | | | ∘ |
| (33-53 μm) + gold | | | | | | | |
| (<32 μm) | | | | | | | |
| Sieved BP-R Long | | | ∘ | | | | ∘ |
| (<32 μm) + gold | | | | | | | |
| (<32 μm) | | | | | | | |
| Sieved BP-R Excellent | | | ∘ | | | | ∘ |
| (<32 μm) + gold | | | | | | | |
| (<32 μm) | | | | | | | |
| Sieved Cerapaste | | | ∘ | | | | ∘ |
| (<32 μm) + gold | | | | | | | |
| (< 32 μm) | | | | | | | |
| Sieved Cerapaste | | | Δ | | | | x |
| (33-53 μm) + gold | | | | | | | |
| (<32 μm) | | | | | | | |

*1: Indicates inner diameter/cross-sectional area

Similarly, for the product manufactured by The Nilaco Corporation (product number: AU-174015: particle size: 1 to 2 μm), a G/CPC paste in which a gold particle fraction sieved using a 32 μm sieve after grinding in a mortar was kneaded together with Biopex-R (Excellent (EX)) sieved in the same manner using a 32 μm sieve also easily passed through a 22 G puncture needle (needle length: 20 cm).

### Example 10

[Particle Size Measurement of Gold Particles]

Pure gold particles prepared from a heated melt of pure gold having a purity of at least 99.99 wt % by the atomizer method were sequentially sieved using sieves having openings (JIS Z 8801) of 150 μm, 75 μm, 53 μm, and 32 μm, and the obtained pure gold particle fractions having particle widths of 150 to 76 μm, 75 to 54 μm, 53 to 33 μm, and 32 μm or less, pure gold particles having a particle width of 20 μm or less obtained by further fractionating the fraction of 32 μm or less, and further, an agate mortar-ground product of pure gold particles manufactured by The Nilaco Corporation, and a fraction of pure gold particles having a particle width of 32 μm or less obtained by further sieving the pure gold particles using a 32 μm sieve, were respectively subjected to a particle size distribution measurement according to the following method.

(1) Experimental Method

The particle size distributions of the pure gold particles were carried out by a wet method using a particle size distribution measurement device manufactured by MicrotracBEL Corporation (MT3000II) and using IPA (isopropyl alcohol) as the dispersion medium, with a measurement frequency of Avg/2 and a measurement time of 30 sec in each case. When the particle size distribution is represented by a cumulative distribution, the side representing the finest particles is expressed as zero, and D50, D10, and D90 and the like are used to represent the distribution. The D50 value is referred to as a median diameter, and represents the diameter at which the larger side and the smaller side are equal, while D10 is the particle size at which the cumulative distribution from the side representing the smallest particles is 10%, and D90 is the particle size at which the cumulative distribution from the side representing the largest particles is 10%. MV represents the volume mean diameter, MN the number average diameter, and MA the area average diameter.

(2) Results

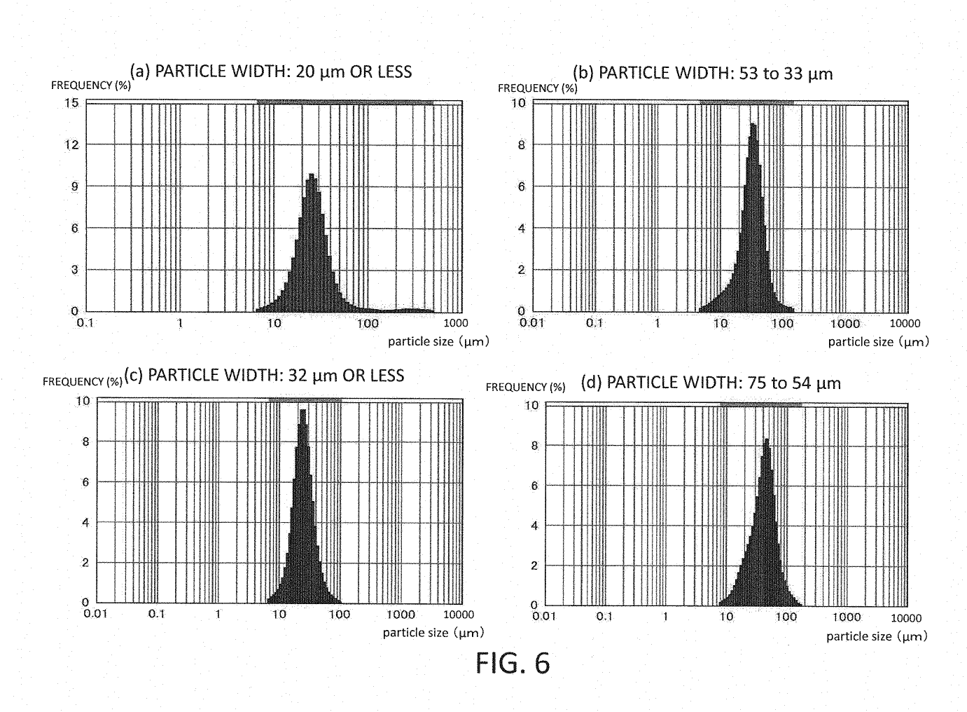

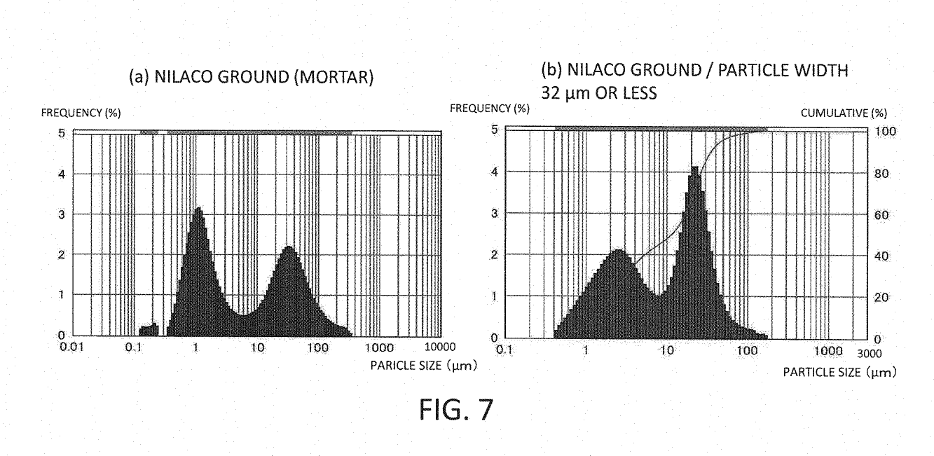

The results are shown in FIG. 6(a), (b), (c), (d), FIG. 7(a), (b), and Table 16.

TABLE 16
Particle size distribution measurement results for various pure gold particles

| Type of gold particle | D50 | D10 | D90 | MV | MN | MA |
|---|---|---|---|---|---|---|
| 1) 20 μm or less | 25.45 | 15.62 | 43.04 | 33.17 | 16.53 | 23.78 |
| 2) 32 μm or less | 24.22 | 14.88 | 40.06 | 26.38 | 15.87 | 22.41 |
| 3) 53 to 33 μm | 31.97 | 15.82 | 52.15 | 34.03 | 11.98 | 26.23 |
| 4) 75 to 54 μm | 41.29 | 19.74 | 70.16 | 44.11 | 19.47 | 34.45 |
| 5) 150 to 76 μm | 109.6 | 53.39 | 139.7 | 103.1 | 14.06 | 73.92 |
| 6) Nilaco (mortar-ground) | 4.026 | 0.699 | 62.92 | 213.59 | 0.260 | 1.688 |
| 7) Nilaco (mortar-ground, 32 μm or less) | 11.14 | 1.248 | 34.78 | 15.95 | 0.823 | 3.463 |

(values in the table represent particle sizes [μm])

From the results above, it is thought that the pure gold particles appropriate for passage through a long, thin diameter needle have a D50 value within a range from 20 to 35 μm, more preferably within a range from 20 to 30 μm, and even more preferably within a range from 22 to 27 μm. Further, it is thought that, for a D50 value in the ranges above, pure gold particles having a D10 value of at least 10 μm and a D90 value of 55 μm or less are preferred, pure gold particles having a D10 value of at least 12 μm and a D90 value of 48 μm or less are more preferred, and pure gold particles with a D50 value in the ranges above and having a D10 value of at least 13 μm and a D90 value of 44 μm or less are particularly preferred. If the D50 exceeds about 40 μm, passage through a 20 G to 22 G puncture needle with a needle length of 20 cm may sometimes become difficult.

Furthermore, using the volume mean diameter (MV) to express preferred pure gold particles, a range from 20 to 38 μm is preferred, a range from 20 to 30 μm is more preferred, and a range from 23 to 29 μm is even more preferred. If the MV of the pure gold particles exceeds about 45 μm, passage through a 20 G to 22 G puncture needle with a needle length of 20 cm may sometimes become difficult.

On the other hand, as shown in Table 16, the D90 value of pure gold particles having a particle width of 32 μm or less (Table 16-2) was 40.06 μm, and a comparison of the cumulative frequency up to a particle size of 40.35 μm, which is in the vicinity thereof, for the pure gold particles 1), 2), 3), 4), 5), and 7) listed in Table 16 above gives 87.82%, 90.30%, 73.44%, 47.86%, 7.47%, and 93.13% respectively. Furthermore, a comparison of the cumulative frequency up to a particle size in the vicinity of 32 μm (31.11 μm) gives 71.33%, 75.08%, 47.20%, 28.50%, 6.14%, and 86.82% respectively. In addition, a comparison of the cumulative frequency up to a particle size in the vicinity of 100 μm (95.96 μm) gives 97.20%, 99.95%, 98.91%, 97.04%, 31.79%, and 99.02% respectively. Therefore, expressed as the cumulative frequency of the particle size distribution, it may be stated that a cumulative frequency (volume ratio) of particles having a particle size of 40 μm or less of at least 70% is preferred, and at least 85% is more preferred, while a cumulative frequency (volume ratio) up to a particle size of about 31 μm of at least 45% is preferred, and at least 70% is more preferred. Furthermore, it was suggested that a cumulative frequency (volume ratio) of particles having a particle size of at least about 96 μm of about 1.5% or less is preferred, about 1% or less is more preferred, and about 0.2% or less is even more preferred. In the pure gold particles, if the abundance ratio of particles having a particle size exceeding about 96 μm exceeds 3%, passage through a 20 G to 22 G puncture needle with a needle length of 20 cm may sometimes become difficult.

### Example 11

[Particle Size Distribution Measurement of CPC Powder]

(1) Experimental Method

The particle size distributions of the CPC powders were measured by a wet method using a laser diffraction and scattering particle size distribution measurement device Microtrac MT 3300EX-II (MicrotracBEL Corporation) and water as the dispersion medium, using a measurement frequency of Avg/3 and a measurement time of 10 sec in each case.

(2) Results

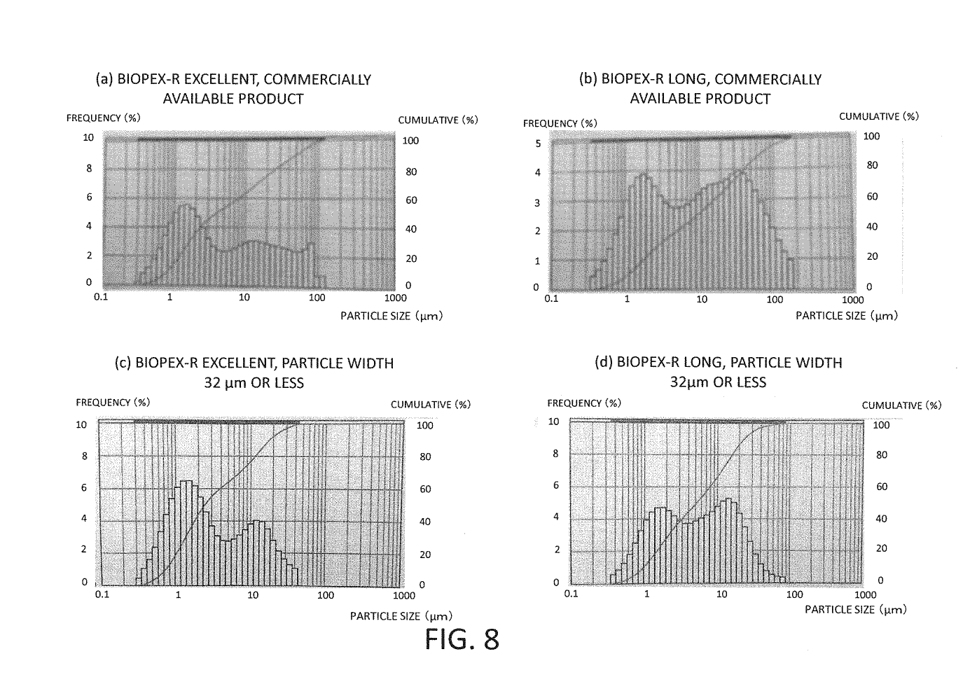

The results are shown in FIG. 8(a), (b), (c). (d), and Table 17.

TABLE 17
Particle size distribution measurement results for various CPCs

| Type of CPC | D10 | D50 | D90 | MV | MN | MA |
|---|---|---|---|---|---|---|
| 1) Biopex-R EX (commercially available product) | 0.820 | 3.819 | 50.03 | 15.27 | 0.646 | 2.113 |
| 2) Biopex-R Long (commercially available product) | 1.110 | 9.592 | 66.79 | 23.52 | 0.706 | 3.167 |
| 3) Sieved Biopex-R EX(<32 μm) | 0.725 | 2.365 | 17.47 | 6.314 | 0.622 | 1.707 |
| 4) Sieved Biopex-R Long (<32 μm) | 0.964 | 5.075 | 22.92 | 9.399 | 0.709 | 2.492 |

(values in the table represent particle sizes [μm])

According to FIG. 8 and Table 17, from the perspective of the volume mean diameter (MV), a CPC appropriate in terms of the passability through a long, thin diameter needle is within a range from 3 to 12 μm, and more preferably within a range from 4 to 8 μm. Furthermore, the D90 value is preferably within a range from 10 to 30 μm, and more preferably within a range from 10 to 20 μm. If the D90 value of the CPC powder exceeds about 60 μm, passage of the G/CPC paste through a 20 G to 22 G puncture needle (needle length: 20 cm) may sometimes become difficult.

As is evident from FIG. 8, CPC powders that have been sieved through a sieve having openings of 32 μm each had a significant reduction in the fraction of particles exceeding a particle size of about 30 to 40 μm compared to the commercially available products. In addition, for the CPCs 1), 2), 3), and 4) in Table 17, the cumulative frequency (volume ratio) of the particle size distribution up to 31.11 μm was 83.14%, 74.96%, 97.67%, and 95.16% respectively. In other words, it was shown that a CPC powder having passability through a thin puncture needle preferably has a distribution (volume ratio) of particles exceeding a particle size of about 31 μm of about 10% or less, more preferably about 6% or less, and even more preferably about 3% or less. In the particle size distribution of the CPC powder, if the abundance ratio (volume ratio) of particles exceeding about 31 μm exceeds 20%, passage of the G/CPC paste through a 20 G to 22 G puncture needle (needle length: 20 cm) may sometimes become difficult.

### Example 12

[Placement Testing in Dog Liver]

Placement testing in live dogs was carried out in order to ascertain whether or not the G/CPC kneaded materials of the present invention rapidly form clusters having satisfactory image recognition performance when injected into the liver of a living body, if the G/CPC markers exist stably for a long period of time, and whether or not there are problems in terms of safety with respect to a living body.

(1) Sample Preparation and Injection Method

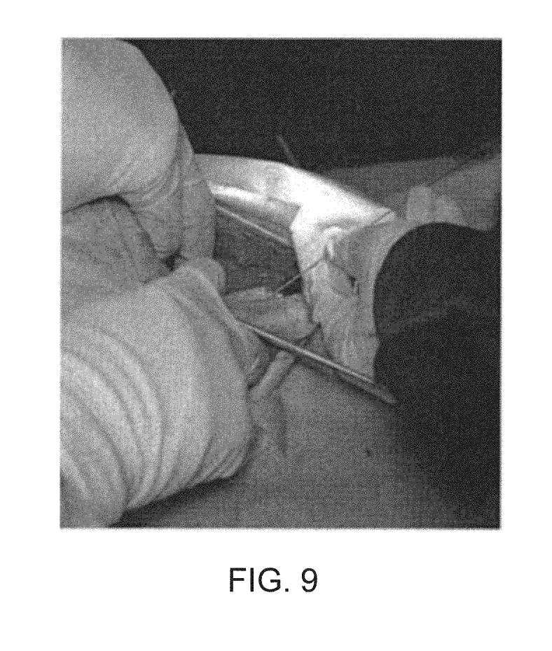

About 3 g of Biopex-R (Standard Type) powder, about 1.5 g of pure gold particles (particle width: 32 μm or less) were mixed in the supplied mortar, and then this mixture was kneaded together with 1.2 mL of a dedicated mixing solution for Biopex-R to prepare a pure gold particle/CPC (referred to as G/CPC below) kneaded material. One mL capacity syringes filled with an appropriate amount of the prepared kneaded material were fitted with a 20 G metallic needle (needle length: about 20 cm), and injection into a liver of a live dog was carried out at room temperature (about 20° C.) (see FIG. 9).

A large amount of the paste (about 0.1 mL×10 times) was injected into the liver of four dogs, and the image recognition performance of the G/CPC markers placed in the liver was investigated on the same day for two of the dogs, and 28 days later for the remaining two dogs, and for the dogs that were observed for 28 days, toxicological evaluation, such as tracking of changes in laboratory values and observation of tissue in the vicinity of the embedded marker, was also performed. The liver was extracted about 1 hour after injection of the G/CPC paste into the dog liver, and after rapid cooling, a portion of the liver was provided for evaluating the existence or absence of marker cluster formation.

Injection into the first dog was carried out with an opened abdomen, and the G/CPC paste was injected under visual control. Percutaneous injection into the liver was attempted for the second dog while referring to an image of a simple ultrasound image device (US) in a state where only the skin had been peeled away, but it was difficult to precisely perform the injection due to problems such as the performance of the US equipment that was used. Injection into both the third and fourth dogs was performed with an opened abdomen, and the same G/CPC paste was injected under visual control.

(2) Evaluation Method and Results

2-1) Cluster Formation of G/CPC Markers Placed in Dog Liver

The first and second dogs were euthanized about 1 hour after injection of the G/CPC paste, and the livers were extracted. Injection into the liver was performed without any problems in the first dog, which was injected under visual control, but CPC was present on the surface of the liver and inside the abdominal cavity of the second dog, which used a simple ultrasound image device, and none was accurately injected into the liver. A portion was injected into the stomach wall and clearly formed clusters, and the fact that this was able to be adequately confirmed by X-ray fluoroscopy strongly suggests that the G/CPC marker of the present invention is capable of being injected and used inside the gastrointestinal wall.

In previous placement testing in removed pig livers, cases where the G/CPC marker cluster formation is not appropriate (formation of small particles) was observed with a high frequency due to, among other reasons, a low liver temperature and a high water content in the injected paste, but for the G/CPC marker placed in a dog liver, cluster formation of about 100 mg was observed in many cases for a placement of about 0.1 mL of the G/CPC paste. However, small clusters were formed in some cases for a portion where reflux of the G/CPC paste occurred from the injection site and the like, although they were all firmly embedded in the liver tissue. As disclosed in PCT International Publication No. WO2016/137013, when about 0.1 mL of the same G/CPC paste is injected into a gel-like composition containing glucomannan (product name: Konnyakubatake; manufactured by Mannanlife Co., Ltd.), or when injection is performed into a pig liver following extraction, significant leakage of the paste from the injection site was not observed as in the present case of injection into the liver of a live dog, and the cause remains unknown.

Similarly, in the dogs after 28 days, about 100 mg of the marker clusters were extracted from the liver, suggesting that the marker clusters that were formed essentially retained their shape over the 28 days.

2-2) Evaluation of Image Recognition Performance of G/CPC Markers Placed in Dog Liver

(Evaluation Method)

The G/CPC paste was injected into the dog liver, and the image recognition performance was evaluated for G/CPC markers placed in the livers extracted about 1 hour after injection, and the livers extracted after 28 days had elapsed from injection. The extracted liver was placed on an acrylic phantom, and an X-ray fluoroscopy device (X-ray generation device: UD150B-40 manufactured by Shimadzu Corporation; flat panel detector (FPD) for X-ray image acquisition: PaxScan 3030 manufactured by Varian Medical Systems, Inc.) was used to acquire X-ray fluoroscopy images. The 1.5 mm and 2.0 mm diameter pure gold spherical markers (iGold) that are currently used clinically were used as positive controls, and in the liver extracted after 28 days, a wire form marker (Gold Anchor) of an alloy containing 99.5 wt % gold and 0.5 wt % iron having a diameter of 0.28 mm and a length of 10 mm and 20 mm was further pasted onto the extracted liver. The acrylic plate thickness was gradually changed from 1 cm to 25 cm, the tube voltage of the X-ray generation device was fixed at 110 kV, the exposure time was fixed at 3 msec, the tube current was selected from 50 mA, 80 mA and 160 mA depending on the situation, and about 100 X-ray fluoroscopy images were acquired under each of the conditions. A template image was created by cutting out an image of the pure gold particle marker targeted for evaluation from one of the plurality of images from among the X-ray fluoroscopy images obtained under each of the conditions, template pattern matching was performed with respect to the other images by normalized cross-correlation with the template image created beforehand, and when the average value of a correlation coefficient obtained from template pattern matching for about 100 images exceeded 0.3, image recognition was determined to be possible, while image recognition was deemed not possible for lower values. An image processing library (Matrox Imaging Library 9 manufactured by Matrox Corporation) was used for image gradation processing and pattern matching.

(Results)

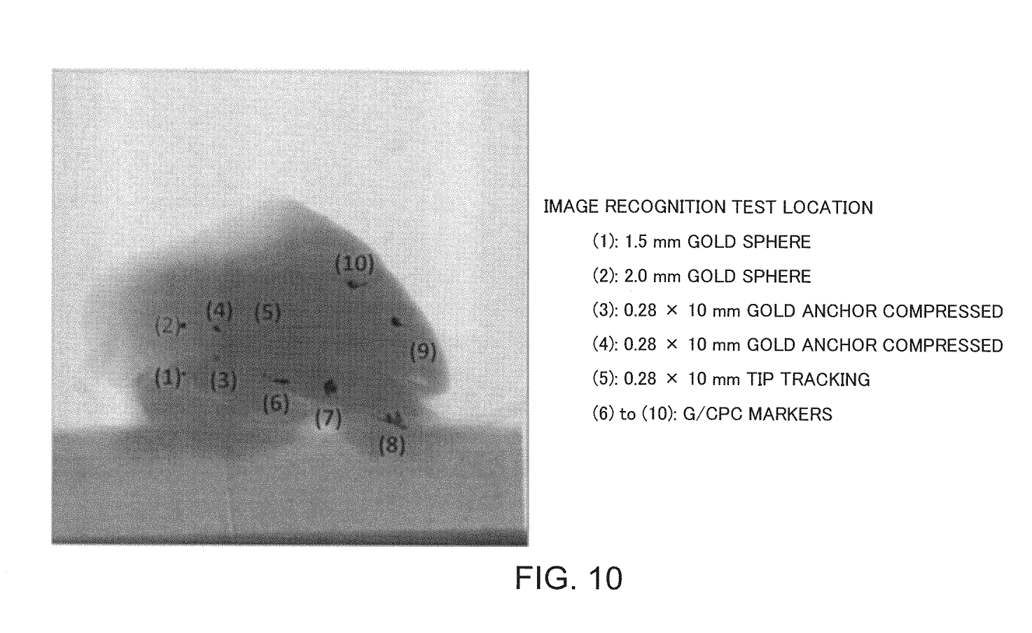

FIG. 10 shows an X-ray fluoroscopy image when the acrylic plate thickness was 1 cm. From a qualitative evaluation by visual inspection of the image, the G/CPC marker was observed to have visibility equivalent to, or better than, the markers used as positive controls. Furthermore, in the evaluation by template pattern matching, image recognition performance equivalent to, or better than, the markers used as positive controls was observed (see Table 18). Furthermore, the same evaluation was performed for a dog liver in which the liver had been injected and then extracted about 1 hour later, and it was confirmed that the G/CPC marker had the same image recognition performance as the G/CPC marker in the liver extracted 28 days later, suggesting that the G/CPC marker of the present invention exists stably for about 1 month inside the liver of a living dog.

TABLE 18
Marker recognition performance in an extracted dog liver after 28 days

| | | Marker | | | | | | | | | |
|---|---|---|---|---|---|---|---|---|---|---|---|
| | | | | (3) | (4) | (5) | | | | | |
| | | | | Gold | Gold | Gold | | | | | |
| | Acrylic | (1) | (2) | Anchor | Anchor | Anchor | | | | | |
| No. | thickness/current | 1.5 mm | 2.0 mm | 10 mm | 20 mm | tip | (6) | (7) | (8) | (9) | (10) |
| 1 | 1 cm/50 mA | 99.2 | 99.3 | 96.7 | 98.4 | 79.1 | 98.7 | 99 | 98.9 | 99.1 | 99 |
| 2 | 5 cm/50 mA | 97.6 | 98 | 88.4 | 96.3 | 56.8 | 96 | 96.8 | 96.7 | 97.3 | 96.6 |
| 3 | 5 cm/80 mA | 98.7 | 99 | 92.8 | 97.6 | 74.6 | 97.5 | 98.4 | 98.1 | 98.7 | 98.2 |
| 4 | 10 cm/50 mA | 90.9 | 91.7 | 63.8 | 85.5 | 8 | 82.7 | 87 | 80.2 | 88.0 | 86.9 |
| 5 | 10 cm/80 mA | 92.9 | 96.3 | 77.8 | 91.8 | 35 | 91.1 | 92.4 | 92.7 | 93.8 | 92.8 |
| 6 | 15 cm/50 mA | 60.7 | 71.2 | 22.4 | 50 | 1.2 | 46.6 | 52.3 | 48.5 | 61.3 | 53.4 |
| 7 | 15 cm/80 mA | 78.8 | 81.3 | 39 | 68.5 | 1 | 64.2 | 68.9 | 67.4 | 74.4 | 67.9 |
| 8 | 20 cm/50 mA | 13.2 | 24 | 1 | 1.2 | 1 | 1 | 1 | 1 | 1.4 | 1.2 |
| 9 | 20 cm/80 mA | 38 | 41.3 | 1.3 | 15.9 | 1 | 10.6 | 6.4 | 6.1 | 28.2 | 15.5 |
| 10 | 20 cm/160 mA | 49.4 | 58.5 | 2.6 | 40.7 | 1 | 30.8 | 34.9 | 35.6 | 47 | 34.6 |
| 11 | 25 cm/80 mA | 2.1 | 1 | 1 | 1 | 1 | 1 | 1 | 1 | 1 | 1 |
| 12 | 25 cm/160 mA | 5.5 | 2.5 | 1 | 1 | 1 | 1 | I | 1 | 1 | 1 |

*The unshaded region represents the region in which kit image recognition was possible

G/CPC marker clusters were extracted from the liver extracted after 28 days that was used in the above test, and upon evaluating the image recognition performance of the G/CPC markers in a similar manner to above, substantially the same result was obtained as the data for a case where the markers were placed inside the extracted liver, suggesting that the G/CPC marker of the present invention is derived from G/CPC markers generated inside the liver.

2-3) Safety Evaluation in Dog with G/CPC Placed Inside Live Liver

Significant bleeding from the liver was not observed for a 20 G puncture needle. For convenience of the operation, the G/CPC paste was injected in a state where the dog abdomen was opened and the liver could be directly observed, and therefore blood and biochemical changes were observed that indicated resulting inflammatory reactions such as a transient increase in CRP and total white blood cells, but it may be stated that these were caused by the above operation.

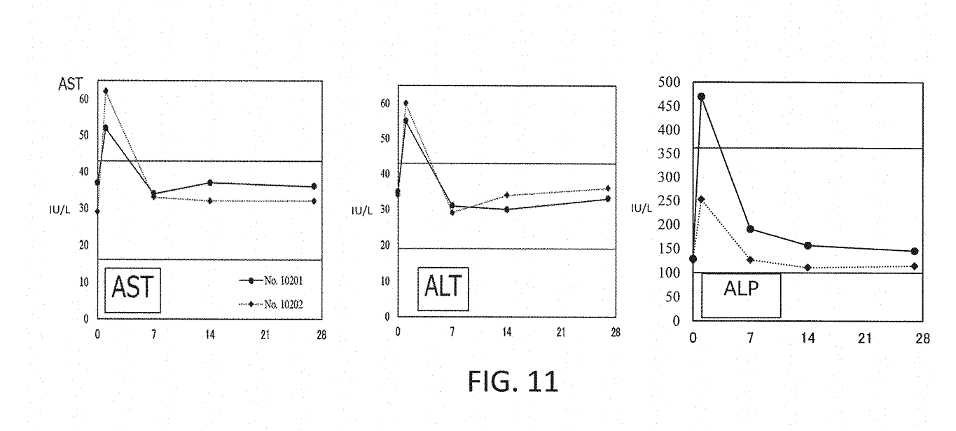

Examples of test value abnormalities reflecting an injury to hepatic parenchymal cells include a transient rise in (AST (GOT): aspartate aminotransferase). (ALT (GPT): alanine aminotransferase), (ALP: Alkaline phosphatase) (see FIG. 11), but given that the increases are transient and the injection amount is much greater than that scheduled to be used clinically (about 30 μL to 50 μL), it may be stated that the placement of the product of the present invention in the liver is toxicologically minor. Pathologically, only a film was formed in the surroundings of the G/CPC marker clusters inside the liver, and irritation and toxicity to the liver cells, such as necrosis, and inflammation and the like, were not observed.

(Summary)

The following objectives, which could not be sufficiently confirmed in previous extracted pig livers and D-PBS(−) circulated extracted pig livers, were able to be confirmed, strongly suggesting that placement is possible as a lesion identification marker for use in radiation therapy in a live body.

(1) Cluster formation in the G/CPC paste of the present invention occurred rapidly when injected into a dog liver, and discharge into the blood stream and discharge of small clusters into the blood stream, which causes pulmonary obstruction and the like, do not occur.

(2) The obtained G/CPC marker clusters have image recognition performance equivalent to existing gold markers, and further, if an increase in the gold particle content in the paste or an increase in the paste placement amount is achieved, demonstration of an enhanced image recognition performance and tracking performance over existing gold markers can be anticipated even with respect to humans, which have a large torso that is difficult for X-ray fluoroscopy. Of course, X-ray fluoroscopy of easier sites can also be handled by reducing the injection amount.

(3) Even after about 1 month, the formed G/CPC marker clusters are present with good retention of tracking performance.

(4) Based on changes in clinical laboratory values and pathological tissue analysis, transient damage to the liver parenchyma is observed directly after injection, but the damage is minor, and a rapid recovery indicates that the possibility of clinical use is high.

As described above, a lesion identification marker for use in radiation therapy and a lesion identification marker kit for use in radiation therapy could be obtained that enable pure gold microparticles which absorb X-rays to be placed, with extremely low invasive potential, in any site in the body in an arbitrary amount appropriate for the type of radiation therapy and the therapeutic target site, and that enable the placement site to be identified over a long period of time by radiation therapy equipment.

### Example 13

[Passability of CPC Pastes or G/CPC Pastes Using Various Sieved Products of CPCs Through a Thin Diameter Needle (2)]

(1) Sieved powders of various commercially available CPCs were prepared again with reference to the results of Example 7 and Example 9, and the passability through a puncture needle (needle length: 20 cm) and a thin diameter needle with a needle length of 3 to 4 cm was investigated for CPC-only pastes and G/CPC pastes. A puncture needle manufactured by Togo Medikit Co., Ltd. and a Create Medic puncture needle (medical device approval number: 201600BZZ00555000) were used as puncture needles (needle length: 20 cm). The inner diameter of the 22 G puncture needle manufactured by Create Medic Co., Ltd. was 0.54 mm, which is substantially equal to that of the 22 G puncture needle (inner diameter: 0.53 mm) manufactured by Togo Medikit Co., Ltd.

(2) Preparation of CPC Sieved Products

Sieved powders of various Biopex products (manufactured by HOYA Technosurgical) and Cerapaste (manufactured by NGK Spark Plug Co., Ltd.) were prepared using the sieves having openings (JIS Z 8801) of 150 μm, 100 μm, 75 μm, 53 μm, 32 μm, 25 μm or 20 μm, and representative results including those from the commercially available products (unsieved) are displayed in Table 19. The sieving was performed using a manual sieving method, by adding 5 to 10 tap balls (polyurethane balls with iron cores ((p 15 mm)) to the upper surface of the sieve, or a method using a sonic sieve (manufactured by Seishin Enterprise Co., Ltd.; GA-8 model). The sieving facilities used were A (a home university facility). B (a commercial facility), and C (a contracted facility).

TABLE 19

| | | | | Sieving |
|---|---|---|---|---|
| Number | CPC product name | Sieve opening | Sieving method | facility |
| C1 | Biopex-R EX | Not sieved | — | — |
| | (commercially available product) | | | |
| C2 | Sieved Biopex-R EX | 75 μm | Tap ball + manual | A |
| C3 | Sieved Biopex-R EX | 53 μm | Tap ball + manual | A |
| C4 | Sieved Biopex-R EX | 32 μm | Tap ball + manual | A |
| C5 | Sieved Biopex-R EX | 32 μm | Tap hall + manual | B |
| C6 | Sieved Biopex-R EX | 32 μm | Tap ball + manual | B |
| C7 | Sieved Biopex-R EX | 32 μm | Tap ball + manual | B |
| C8 | Sieved Biopex-R EX | 32 μm* | Tap ball + manual | B |
| C9 | Sieved Biopex-R EX | 32 μm | Sonic | C |
| C10 | Sieved Biopex-R EX | 32 μm* | Sonic | B |
| C11 | Sieved Biopex-R EX | 25 μm | Sonic | C |
| C12 | Sieved Biopex-R EX | 20 μm | Sonic | C |
| C13 | Biopex-R Long | Not sieved | — | — |
| | (commercially available product) | | | |
| C14 | Cerapaste | Not sieved | — | — |
| | (commercially available product) | | | |
| C15 | Sieved Cerapaste | 32 μm | Tap ball + manual | A |

*These powders were obtained by respectively sieving the five types of components of Biopex-R Excellent (EX) using a 32 μm sieve, and then mixing each of the obtained substances in the composition ratio of Biopex-R.
**Tap ball: 5 to 10 polyurethane balls (φ 15) with an iron core were placed on the upper surface of the sieve.

(3-1) Preparation of CPC Pastes, and Passability Through Thin Diameter Needle

To the various CPC powders listed in Table 19 was added 0.4 mL of a dedicated mixing solution per gram of the CPC in the case of Biopex-R. or 0.45 mL of a hardening liquid per gram of the CPC in the case of Cerapaste, the resulting paste was kneaded at room temperature, and the passability through 22 G to 25 G thin diameter needles (needle length: 3 to 4 cm; inner diameter: about 0.5 mm (22 G), about 0.35 mm (23 G), about 0.3 mm (24 G), about 0.25 mm (25 G)) within 5 min from the start of kneading was investigated (Table 20). In other words, commercially available CPC products other than Cerapaste could not pass through thin diameter needles 23 G and thinner, but in particular, powders sieved with a sieve of 32 μm or less could sometimes pass through a 25 G thin diameter needle provided the needle was short (needle length: 3 to 4 cm).

TABLE 20

| | | Sieve opening/ | Thin diameter needle passability | | | |
|---|---|---|---|---|---|---|
| Number | CPC product name | production method | 22G | 23G | 24G | 25G |
| C1 | Biopex-R EX | Not sieved | x∘x | x | x | x |
| | (commercially available product) | | | | | |
| C2 | Sieved Biopex-R EX | 75 μm/manual | — | — | ∘ | x |
| C3 | Sieved Biopex-R EX | 53 μm/manual | — | — | ∘ | x |
| C4 | Sieved Biopex-R EX | 32 μm/manual | — | — | ∘ | ∘ |
| C5 | Sieved Biopex-R EX | 32 μm/manual | — | — | ∘ | ∘ |
| C6 | Sieved Biopex-R EX | 32 μm/manual | — | — | ∘ | ∘ |
| C7 | Sieved Biopex-R EX | 32 μm/manual | — | — | ∘ | ∘ |
| C8 | Sieved Biopex-R EX | 32 μm/manual* | — | — | — | — |
| C9 | Sieved Biopex-R EX | 32 μm/sonic | — | — | ∘ | ∘ |
| C10 | Sieved Biopex-R EX | 32 μm/sonic* | — | — | — | — |
| C11 | Sieved Biopex-R EX | 25 μm/sonic | — | — | ∘ | ∘ |
| C12 | Sieved Biopex-R EX | 20 μm/sonic | — | — | ∘ | ∘ |
| C13 | Biopex-R Long | Not sieved | ∘ | x | x | x |
| | (commercially available product) | | | | | |
| C14 | Cerapaste | Not sieved | ∘ | ∘ | ∘ | x |
| | (commercially available product) | | | | | |
| C15 | Sieved Cerapaste | 32 μm/manual | — | — | ∘ | x |

*These powders were obtained by respectively sieving the five types of components of Biopex-R Excellent (EX) using a 32 μm sieve, and then mixing each of the obtained substances in the composition ratio of Biopex-R.

(3-2) Preparation of G/CPC Pastes, and Passability Through Puncture Needle (Needle Length: 20 cm)

In Table 15 of Example 9, it was shown that a kneaded material (paste form), in which various CPCs sieved using a 32 μm sieve and gold particles similarly sieved using a 32 μm sieve in a ratio of CPC (g):gold particles (g)=2:1 (weight ratio) were kneaded together with a dedicated mixing solution added in a ratio of CPC (g):dedicated mixing solution (mL)=1:0.4 (weight:volume), was able to pass through a 22 G puncture needle (outer diameter: about 0.7 mm inner diameter: about 0.5 mm; thinnest needle among commercially available disposable puncture needles) with a needle length of 20 cm. Therefore, as listed in Table 19, in the examples of the present invention, various CPC powders were prepared using substantially the same or different conditions to those mentioned above (Table 15), to each gram of the CPC was added 0.5 g of gold particles (32 μm sieved product) and a further 0.4 mL of a dedicated mixing solution in the case of Biopex-R, or 0.45 mL of a hardening liquid in the case of Cerapaste, each mixture was kneaded at room temperature (about 25° C.), and the passability of the kneaded material (paste form) through a 22 G puncture needle (needle length: 20 cm; manufactured by Create Medic Co., Ltd. or Togo Medikit Co., Ltd.) within about 5 min from the start of kneading was reinvestigated. Table 21 shows the results for the case where the puncture needle (needle length: 20 cm) manufactured by Create Medic Co., Ltd. was used, but similar results were obtained in the case of the 22 G puncture needle (needle length: 20 cm) manufactured by Togo Medikit Co., Ltd. Although the data is not specifically shown in Table 21, G/CPC pastes using the commercially available Biopex-R Excellent or the sieved product from a 150 μm sieve both could pass through an 18 G puncture needle (needle length: 20 cm; inner diameter: 1.07 mm), but could not pass through thinner puncture needles, and similarly, a G/CPC paste using a sieved product from a 100 μm sieve could pass through up to a 21 G puncture needle (needle length: 20 cm; inner diameter: 0.59 mm), but could not pass through a 22 G puncture needle. Furthermore, the same passability through a puncture needle was also investigated in CPC-only kneaded materials (CPC-only pastes not containing gold particles), confirming that they show substantially the same passability through a puncture needle as the cases that include gold particles sieved using a 32 μm sieve.

In other words, in the case of Biopex-R, when a sieved product that had been sieved through a sieve having openings of 75 μm to 20 μm was used, passage through a 22 G puncture needle (needle length: 20 cm) was possible irrespective of the presence or absence of gold particles that had been sieved using a 32 μm sieve, and further, a sieved product of Biopex-R Excellent from a 32 μm sieve could also pass substantially through a 25 G thin diameter needle (needle length: 3 to 4 cm). Moreover, a powder in which the components of Biopex-R were each fractionated beforehand using a 32 μm sieve (case C10 in Table 23) or a 25 μm sieve, and then mixed in the same component formulation as Biopex-R, was also able to pass through a 22 G puncture needle (needle length: 20 cm) in a similar manner. Furthermore, the sieved product of Cerapaste from a 32 μm sieve could similarly also pass through a 22 G puncture needle (needle length: 20 cm). Although not shown in the data, even when 1 g of 32 μm sieved gold particles, or 1 g of gold particles having a particle width of 53 to 33 μm (the fraction that passed through the 53 μm sieve but did not pass through the 33 μm sieve) per gram of the sieved CPC from a 32 μm sieve was kneaded together with the same mixing solution ratio as described above, passage through a 22 G puncture needle (needle length: 20 cm) was possible.

TABLE 21

| | | | | 22 G puncture needle |
|---|---|---|---|---|
| | | | | (20 cm) passability |
| | | Sieve opening/ | | Gold particles (32 |
| Number | CPC product name | production method | | μm sieved product) |
| C1 | Biopex-R EX | Not sieved | | x |
| | (commercially available product) | | | |
| C2 | Sieved Biopex-R EX | 75 | μm/manual | ∘ |
| C3 | Sieved Biopex-R EX | 53 | μm/manual | ∘ |
| C4 | Sieved Biopex-R EX | 32 | μm/manual | ∘ |
| C5 | Sieved Biopex-R EX | 32 | μm/manual | ∘ |
| C6 | Sieved Biopex-R EX | 32 | μm/manual | ∘ |
| C7 | Sieved Biopex-R EX | 32 | μm/manual | ∘ |
| C8 | Sieved Biopex-R EX | 32 | μm/manual* | ∘ |
| C9 | Sieved Biopex-R EX | 32 | μm/sonic | ∘ |
| C10 | Sieved Biopex-R EX | 32 | μm/sonic* | ∘ |
| C11 | Sieved Biopex-R EX | 25 | μm/sonic | ∘ |
| C12 | Sieved Biopex-R EX | 20 | μm/sonic | ∘ |
| C13 | Biopex-R Long | Not sieved | | x |
| | (commercially available product) | | | |
| C14 | Cerapaste | Not sieved | | x |
| | (commercially available product) | | | |
| C15 | Sieved Cerapaste | 32 | μm/manual | ∘ |

*These powders were obtained by respectively sieving the five types of components of Biopex-R Excellent (EX) using a 32 μm sieve, and then mixing each of the obtained components in the formulation found in Biopex-R.

### Example 14

[Viscosity of Various CPC Kneaded Materials]

(1) Experimental Method

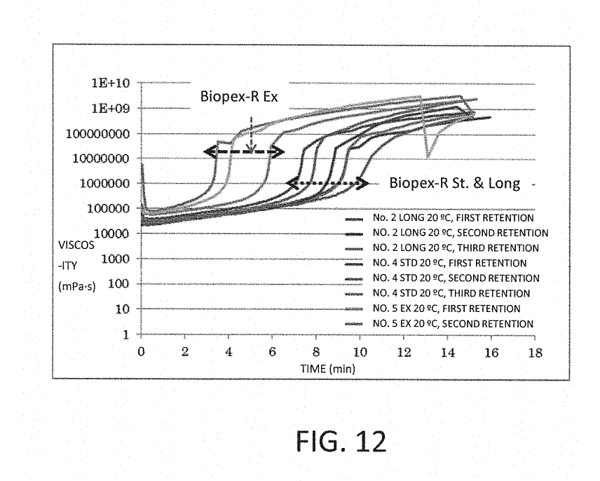

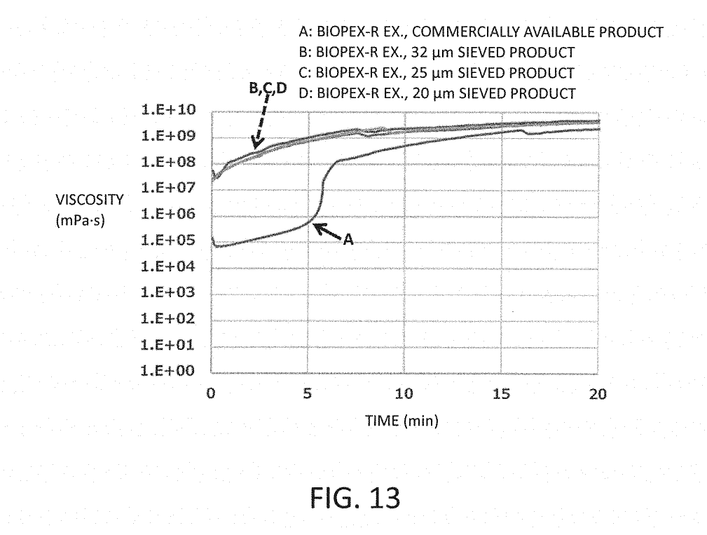

The mixing solution was added to the various CPCs in the amounts listed in Table 22, and after kneading (at about 24° C.) for about 30 sec, about 0.6 mL of the kneaded material was isolated and the viscosity measurement was started about 2 min from the start of kneading (0 min in FIG. 12 and FIG. 13 represents the point at which 2 min had elapsed from the start of kneading). During the measurement, the viscosity measurement was performed while maintaining the sample chamber temperature of the rheometer at 20° C. The viscosity measurements used a RS-600 RheoStress manufactured by Haake GmbH.

(2) Results

Table 22 below shows the viscosity (mPa·s) after about 5 min from the start of kneading.

TABLE 22

| | | | Amount of | |
|---|---|---|---|---|
| | | | mixing solution | |
| | CPC product | | added (per | Viscosity |
| Symbol | name | Sieve opening | gram of CPC) | (mPa · s) |
| P1 | Biopex-R Long | (commercially | 0.4 mL | 10⁴ to 10⁶ |
| | | available | | |
| | | product) | | |
| P2 | Biopex-R | (commercially | 0.4 mL | 10⁴ to 10⁶ |
| | Standard | available | | |
| | | product) | | |
| P3 | Biopex-R EX | (commercially | 0.4 mL | 10⁴ to 10⁶ |
| | | available | | |
| | | product) | | |
| P4 | Sieved Biopex- | 32 μm | 0.4 mL | 10⁸ to 10¹⁰ |
| | R EX | | | |
| P5 | Sieved Biopex- | 25 μm | 0.4 mL | 10⁸ to 10¹⁰ |
| | R EX | | | |
| P6 | Sieved Biopex- | 32 μm | 0.5 mL | about 10⁸ |
| | R EX | | | |
| P7 | Sieved Biopex- | 75 μm | 0.4 mL | 10⁴ to 10⁶ |
| | R EX | | | |

When Biopex-R Excellent Type (Biopex-R EX), Long Type, and Standard Type (Biopex-R St. & Long) are compared, the initial rise in the viscosity is different, with the former displaying a rise in the viscosity from about 3 to 5 min after the start of the viscosity measurement, while the viscosity remains about 10⁶ mPa·s or less in the Long Type and the Standard Type until about 6 to 10 min after the start of the viscosity measurement, and shows a slower initial rise in the viscosity in which a time of about 8 min is required after the start of the measurement (about 10 min after the start of kneading) for the viscosity to reach about 10⁸ mPa·s (FIG. 12). Furthermore, similarly for Cerapaste, a kneaded material in which 0.45 mL of a mixing solution for Cerapaste is used per gram of the CPC showed a transition in the viscosity similar to the commercially available product Biopex-R Excellent Type, and the viscosity at 20° C. about 5 min after the start of kneading was 10⁴ to 10⁶ mPa·s. On the other hand, in P4 (Biopex-R Excellent, 32 μm sieved product) and P5 (Biopex-R Excellent, 25 μm sieved product) in Table 22 and the like, the viscosity at the start of the viscosity measurement was already 10⁷ to 10 mPa·s, and reached an extremely high viscosity of 10 to 10¹⁰ mPa·s about 5 min after the start of kneading (FIG. 13).

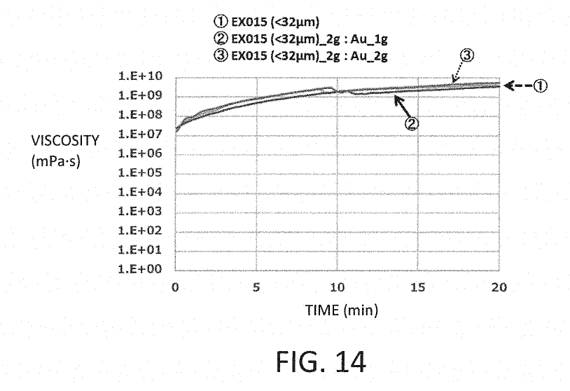

Even at such high viscosities, passage through a 22 G puncture needle (needle length: 20 cm) was possible, and furthermore, when injected into a site with a high tissue pressure such as the liver of a live dog, a remarkable leakage from the placement site was observed when a low-viscosity kneaded material (kneaded material of Biopex-R Standard and gold particles) was used (Example 12), but in the case gold particles mixed under the conditions of P4 in Table 22, leakage of the G/CPC kneaded material from the placement site was essentially not observed (see Example 17). Kneaded materials using a CPC under the same conditions as P4, in which 1 g or 2 g of gold particles (32 μm sieved product) and 0.4 mL of a mixing solution are respectively added to 2 g of the CPC, each had a viscosity of 10⁸ to 10¹⁰ mPa·s about 5 min after the start of kneading, which represents substantially the same viscosity as that of CPC kneaded materials under the conditions of P4 that do not include gold particles (FIG. 14). When the amount of the mixing solution per gram of the CPC was increased from 0.4 mL to 0.5 mL, the viscosity clearly decreased as observed for P6. Upon performing leakage testing at 37° C. by using a commercially-available high-hardness gel (human skin gel (hardness: 5); manufactured by Exseal Co., Ltd.) in the CPC paste, a clear increase in leakage was observed for the P6 paste compared to P4 (kneaded material using 0.4 mL of the mixing solution). The Biopex-R that had been sieved using a 75 μm sieve shown in P7 displayed substantially the same viscosity as the commercially available product (10⁴ to 10⁶ mPa·s), and the distribution of particle sizes exceeding about 31 μm and about 52 μm in the particle size distribution was essentially the same as the commercially available product. Consequently, from the perspective of the passability through a puncture needle with a needle length of 20 cm, it was thought that sieved products having a particle width of 75 μm or less (a powder sieved using a 75 μm sieve) were preferable, but when the viscosity of the paste was also considered, it was thought that powders having a finer particle size distribution than a particle width of 75 μm were preferred. In the leakage testing at 37° C. using a high-hardness gel (human skin gel (hardness: 5); manufactured by Exseal Co., Ltd.), the gel was prepared so as to reach a height of 3 to 4 cm in a plastic container having a diameter of about 4 cm, and about 0.1 mL of the CPC paste or the G/CPC paste of the present invention was injected into the gel, which was maintained at 37° C. beforehand in an incubator, and the determination was made by investigating the amount of leakage about 5 min after injection. In the case of the commercially available Biopex-R, about 20% leakage or more was observed in a large portion of the product. In particular, leakage exceeding about 30% was observed in Biopex-R Standard Type and Biopex-R Long Type, suggesting that there was a correlation between the viscosity transition in the paste and the ease of leakage from the gel.

Based on these observations, in the case of Biopex-R, it was thought a kneaded material under the conditions of P4, in which the viscosity becomes 10 to 10¹⁰ mPa·s about 5 min after the start of kneading, was most preferable for use as a base material for the G/CPC marker. Although the data is not specifically shown, in CPCs prepared under the conditions of C10 in Table 19, or in CPC powders prepared under the same conditions as C10 except for the openings of the sieve being 25 μm, the kneaded materials prepared using the same quantity of the mixing solution as P4 had an equivalent viscosity to P4 or P5. Furthermore, from the perspective of the viscosity of the paste, an addition amount of the mixing solution of 0.3 mL or less per gram of the CPC led to the observation of a trend in which degassing was difficult, and addition of 0.5 mL gave a viscosity of 10⁸ mPa·s about 5 min after the start of kneading, indicating that 0.3 mL to 0.5 mL per gram of the CPC was preferable.

When a sieved product of Biopex-R Excellent from a 20 μm sieve is compared to one that has been fractionated using a 32 μm or 25 μm sieve, considering that there is a trend toward observation of a reduced fractionation yield, and that the removal of air from inside the kneaded material sometimes does not proceed smoothly, it was thought that a sieved product from openings of at least 20 μm, preferably at least 25 μm, or more preferably at least 32 μm, was preferable.

### Example 15

[Particle Size Distribution of Various CPC Sieved Products (2)]

(1) Experimental Method

In the same manner as Example 11, the particle size distributions of the various CPC powders were measured by a wet method using a laser diffraction and scattering particle size distribution measurement device Microtrac MT 3300EX-II (MicrotracBEL Corporation) and water as the dispersion medium, using a measurement frequency of Avg/3 and a measurement time of 10 sec in each case.

(2) Results

The results are shown in Table 23 below, together with Table 17.

TABLE 23

| | | | | Summary data of particle size | | | |
|---|---|---|---|---|---|---|---|
| | | Sieve | Sieving | distribution (unit: μm) | | | |
| Number | Type of CPC | opening | facility | D10 | D50 | D90 | MV |

Table 17 portion

| | | | | | | | |
|---|---|---|---|---|---|---|---|
| 1) | Biopex-R EX | None | | 0.82 | 3.819 | 50.03 | 15.27 |
| | (commercially available product) | (EX007) | | | | | |
| 2) | Biopex-R Long | None | | 1.11 | 9.592 | 66.79 | 23.52 |
| | (commercially available product) | (BL011) | | | | | |
| 3) | Sieved Biopex-R EX | 32 μm | A | 0.725 | 2.365 | 17.47 | 6.314 |
| 4) | Sieved Biopex-R Long | 32 μm | A | 0.964 | 5.075 | 22.92 | 9.399 |

Additional portion

| | | | | | | | |
|---|---|---|---|---|---|---|---|
| C1 | Biopex-R EX | None | | 0.828 | 5.282 | 69.86 | 20.47 |
| | (commercially available product) | (EX020) | | | | | |
| C2 | Sieved Biopcx-R EX | 75 μm | A | 0.797 | 3.987 | 39.57 | 12.64 |
| C3 | Sieved Biopex-R EX | 53 μm | A | 0.775 | 2.913 | 28.37 | 9.777 |
| C4 | Sieved Biopex-R EX | 32 μm | A | 0.73 | 2.375 | 17.61 | 6.332 |
| C5 | Sieved Biopex-R EX | 32 μm | B | 0.711 | 2.348 | 18.36 | 6.483 |
| C6 | Sieved Biopex-R EX | 32 μm | B | 0.701 | 2.268 | 18.11 | 6.368 |
| C7 | Sieved Biopex-R EX | 32 μm | B | 0.742 | 2.388 | 18.1 | 6.422 |
| C8 | Sieved Biopex-R EX | 32 μm | B | 0.73 | 2.516 | 19.22 | 6.794 |
| C9 | Sieved Biopex-R EX | 32 μm | C | 0.749 | 2.3765 | 21.91 | 7.564 |
| C10 | Sieved Biopex-R EX | 32 μm | B | 0.788 | 3.605 | 25.64 | 8.979 |
| C11 | Sieved Biopex-R EX | 25 μm | C | 0.79 | 2.733 | 19.15 | 6.843 |
| C12 | Sieved Biopex-R EX | 20 μm | C | 0.737 | 2.245 | 13.93 | 5.162 |
| C13 | Biopex-R Long | None | | — | — | — | — |
| C14 | Cerapaste | None | | 0.789 | 8.09 | 39.26 | 14.48 |
| | (commercially available product) | | | | | | |
| C15 | Sieved Cerapaste | 32 μm | A | 0.778 | 8.973 | 24.78 | 10.82 |

(values in the table represent particle sizes (μm))

In Table 23 above, the sieved products (C4 to C 10) obtained using a 32 μm sieve had a standard deviation of the MV value of about 1 μm. Therefore, when setting the preferred range for the MV value, it is desirable to add or subtract approximately two to three standard deviations from the measured value.

From the results of Examples 9, 11, 13, 14, and 15, the CPC appropriate in terms of the passability of the G/CPC kneaded material of the present invention through a long puncture needle, from the perspective of the volume mean diameter (MV), is within a range from 3 to 12 μm, more preferably within a range from 4 to 12 μm, even more preferably within a range from 5 to 11 μm, and particularly preferably within a range from 5 to 10 μm. Furthermore, the D90 value is preferably less than 39 μm, more preferably 34 μm or less, even more preferably 30 μm or less, and particularly preferably within a range from 10 to 30 μm. If the D90 value of the CPC powder exceeds about 50 μm, passage of the G/CPC paste through a 20 G to 22 G puncture needle (needle length: 20 cm) may sometimes become difficult.

As is evident from FIG. 8, CPC powders that had been sieved through a sieve having openings of 32 μm each had a significant reduction in the fraction of particles exceeding a particle size of about 30 to 40 μm compared to the commercially available products. In addition, for the CPCs 1), 2), 3), and 4) in Table 17, the cumulative frequency (volume ratio) of the particle size distribution up to 31.11 μm was respectively 83.14%, 74.96%, 97.67%, and 95.16%. In other words, it was shown that a CPC powder having passability through a thin puncture needle preferably has a distribution (volume ratio) of particles exceeding a particle size of about 31 μm of about 15% or less, and more preferably about 10% or less. In the particle size distribution of the CPC powder, if the abundance ratio (volume ratio) of particles exceeding about 31 μm exceeds 15%, passage of the G/CPC paste through a 20 G to 22 G puncture needle (needle length: 20 cm) may sometimes become difficult.

### Example 16

[Passability of G/CPC Pastes Using Various Sieved Products of Gold Particles Through a Thin Diameter Needle (2) and Particle Size Distribution of Gold Particles]

(1) Based on the results of Examples 7, 8, and 9, a sieved powder (32 μm sieved product) of the commercially available product Biopex-R Excellent and various gold particles having different sieving conditions were again kneaded together, and the passability of the obtained pastes through a puncture needle (needle length: 20 cm), and the passability through a short, thin diameter needle were investigated. The Create Medic puncture needle (medical device approval number: 201600BZZ00555000) used in Example 9 was used as the puncture needle (needle length: 20 cm). The inner diameter of the 22 G puncture needle manufactured by Create Medic Co., Ltd. was 0.54 mm, which is substantially equal to that of the 22 G puncture needle manufactured by Togo Medikit Co., Ltd.

(2) Preparation of Kneaded Materials

Gold particles having the respective particle widths of 75 to 54 μm, 53 to 33 μm, 32 to 21 μm, 32 μm or less, and 20 μm or less were each prepared by sequential sieving using sieves having sieve openings of 150 μm, 75 μm, 53 μm, 32 μm, and 20 μm. The pastes were prepared by adding 0.5 g of the particles, 1 g of Biopex-R Excellent that had been passed through a 32 μm sieve, and 0.4 mL of a dedicated mixing solution for Biopex-R, and the needle passability properties of the obtained G/CPC kneaded materials (paste form) were again compared.

(3) Particle Size Distribution Measurement of Various Gold Particles

As described in Example 10, the particle size distributions of the various gold particles were measured by a wet method using a particle size distribution measurement device manufactured by MicrotracBEL Corporation (MT3000II) and IPA (isopropyl alcohol) as the dispersion medium, allowing the D50, D10, and D90 and the like to be determined.

(4) Results

As shown in FIG. 4 and FIG. 5, because fine gold particles have a tendency to form aggregated clusters, as shown in FIG. 8, almost no reduction in the D50 value was observed in gold particles sieved using a 20 μm sieve, while a clear increase in particles exceeding 100 μm was observed, and when a 32 μm sieved product of Biopex-R Excellent was used as the CPC, gold particles having a particle width of 20 μm or less and a particle width of 75 to 54 μm sometimes passed through a 22 G puncture needle (see Table 24), and therefore gold particles appropriate for passage through a puncture needle (needle length: 20 cm) have a D50 value preferably within a range from 16 to 40 μm, more preferably within a range from 18 to 36 μm, and even more preferably within a range from 20 to 35 μm. Further, it is thought that, with a D50 value in the ranges above, a D10 value of at least 5 μm and a D90 value of 70 μm or less is preferred, a D10 value of at least 7 μm and a D90 value of 60 μm or less is more preferred, and a D10 value of at least 10 μm and a D90 value of 55 μm or less is even more preferred. If the D50 exceeds about 40 μm, passage through a 20 G to 22 G puncture needle with a needle length of 20 cm may sometimes become difficult.

Furthermore, using the volume mean diameter (MV) to express the preferred pure gold particles, a range from 17 to 44 μm is preferred, a range from 17 to 38 μm is more preferred, and a range from 20 to 38 μm is even more preferred. If the MV of the pure gold particles exceeds about 45 μm, passage through a 20 G to 22 G puncture needle with a needle length of 20 cm may sometimes become difficult.

On the other hand, as shown in Table 16, the D90 value of pure gold particles having a particle width of 32 μm or less (Table 16-2) was 40.06 μm, and a comparison of the cumulative frequency up to a particle size of 40.35 μm, which is in the vicinity thereof, for the pure gold particles 1), 2), 3), 4), 5), and 7) listed in Table 16 above gives 87.82%, 90.30%, 73.44%, 47.86%, 7.47%, and 93.13% respectively. Furthermore, a comparison of the cumulative frequency up to a particle size in the vicinity of 32 μm (31.11 μm) gives 71.33%, 75.08%, 47.20%, 28.50%, 6.14%, and 86.82% respectively. In addition, a comparison of the cumulative frequency up to a particle size in the vicinity of 100 μm (95.96 μm) gives 97.20%, 99.95%, 98.91%, 97.04%, 31.79%, and 99.02% respectively, or in other words, the distribution of particle sizes exceeding a particle size of about 96 μm was 2.8%, 0.05%, 1.09%, 2.96%, 68.21%, and 0.98% respectively. Therefore, expressed as the cumulative frequency of the particle size distribution, it may be stated that a cumulative frequency (volume ratio) of particles having a particle size of 40 μm or less of at least 50% is preferred, at least 65% is more preferred, at least 70% is even more preferred, and at least 85% is particularly preferred, while a cumulative frequency (volume ratio) up to a particle size of about 31 μm of at least 30% is preferred, at least 35% is more preferred, and at least 70% is even more preferred. In the pure gold particles, if the abundance ratio of particles having a particle size exceeding about 96 μm exceeds about 3%, passage through a 20 G to 22 G puncture needle with a needle length of 20 cm may sometimes become difficult.

TABLE 24

| | | | | | | | | | Puncture | Passability | |
|---|---|---|---|---|---|---|---|---|---|---|---|
| | Particle | | | | | | | | needle | through | |
| | width | | | | | | | | passability | thin diameter | |
| | of gold | | | | | | | | (20 cm) | needle (3-4 cm) | |
| | particles | | | | | Particle size distribution (%) | | | Sieved | Sieved | |
| | (classified | Particle size of gold particles (μm) | | | | | 40.35 or | 31.11 or | BIOPEX-R | BIOPEX-R | |
| | by sieving | D50 | D10 | D90 | | >95.96 | less | less | EX (*) | EX (*) | |
| | conditions) | value | value | value | MV | (μm) | (nm) | (μm) | 22G | 24G | 25G |
| 1 | 20 μm or less | 25.45 | 15.62 | 43.04 | 33.14 | 2.8 | 87.82 | 71.33 | ∘ | ∘ | Δ/x |
| 2 | 32-21 μm | 27.21 | 17.46 | 45.24 | 31.07 | 1.91 | 85.61 | 65.74 | ∘ | ∘ | Δ/x |
| 3-1 | 32 μm or less | 24.22 | 14.88 | 40.06 | 26.38 | 0.05 | 90.3 | 75.08 | ∘ | — | — |
| 3-2 | 32 μm or less | 20.46 | 10.88 | 32.77 | 21.6 | 0 | 95.93 | 87.6 | ∘ | — | — |
| 3-3 | 32 μm or less | 20.91 | 10.25 | 36.45 | 22.78 | 0.05 | 93.14 | 82.06 | ∘ | ∘ | ∘ |
| 3-4 | 32 μm or less | 20.84 | 9.763 | 36.05 | 22.45 | 0 | 93.54 | 83.01 | ∘ | — | — |
| 3-5 | 32 μm or less | 24.66 | 13.82 | 39.68 | 26.16 | 0 | 90.75 | 74.1 | ∘ | — | — |
| 4 | 53-33 μm | 31.97 | 15.82 | 52.15 | 34.03 | 1.09 | 73.44 | 47.2 | ∘ | ∘ | Δ/Δ |
| 5 | 75-54 μm | 41.29 | 19.74 | 70.16 | 44.11 | 2.96 | 47.86 | 28.5 | Δ/∘ | ∘ | x |
| 6 | 150-76 μm | 109.6 | 53.39 | 139.7 | 103.1 | 68.71 | 7.47 | 6.14 | x | x | x |

### Example 17

[Placement Testing in Dog Liver and the Like (2)]

Placement testing in live dogs was carried out again with an opened abdomen in order to ascertain whether or not the G/CPC kneaded materials of the present invention rapidly form clusters corresponding to the injection amount when injected into the liver of a living body or the like, whether these clusters have an adequate image recognition performance, and further, if the G/CPC markers exist stably for a long period of time, and whether or not there are problems in terms of the safety with respect to a living body. Here, the target placement organs were made the liver, the pancreas, and the stomach.

(1) Sample Preparation and Injection Method

About 2 g of Biopex-R Excellent (particle width: 32 μm or less), about 1.0 g of pure gold particles (particle width: 32 μm or less) were mixed in the supplied mortar, and then this was kneaded together with 0.8 mL of a dedicated mixing solution for Biopex-R to prepare the pure gold particle/CPC (referred to as G/CPC below) kneaded material (also referred to as a G/CPC paste). One mL capacity syringes filled with an appropriate amount of the prepared kneaded material were fitted with a 22 G metallic needle (needle length: about 20 cm), and about 0.1 mL according to the syringe scale was injected into the liver, pancreas, and stomach of a dog live body at room temperature (about 20 to 26° C.).

Of the eight dogs, two dogs were used as a control group in which the G/CPC paste from above was not injected, and only the opening and closing of the abdomen was performed before completion. Thereafter, for the remaining six dogs, injection was carried out with respect to the liver for two of the dogs, the pancreas for two of the dogs, and the stomach for two of the dogs, with about five locations injected in each dog (about 0.1 mL of the paste in each case). Injection into the six dogs was carried out with an open abdomen, and the G/CPC paste was injected under visual control.

The six dogs were each subjected to investigation of the image recognition performance of the G/CPC markers placed in the liver, inside the stomach wall, and in the pancreas 28 days later, the weight of the extracted marker clusters was investigated, and toxicological evaluation, such as tracking of changes in clinical laboratory values, and observation of tissue in the vicinity of the embedded marker and the like, was also performed over the 28 days.

(2) Evaluation Method and Results

2-1) Cluster Formation for G/CPC Markers Placed in Dog Liver, Stomach Wall, and Pancreas

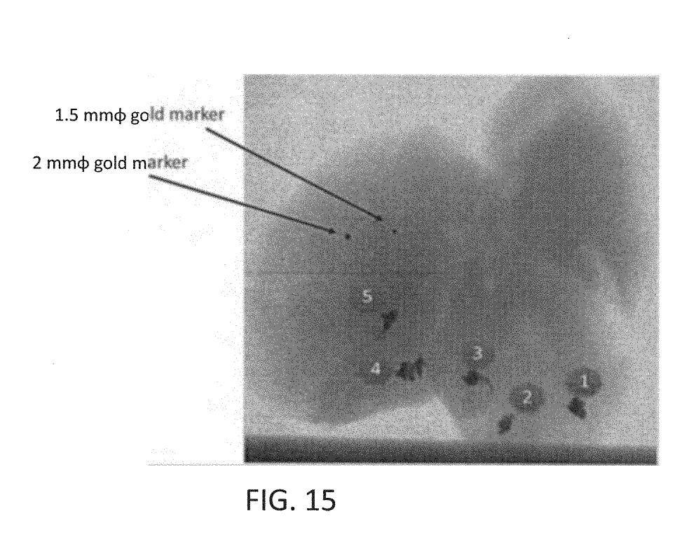

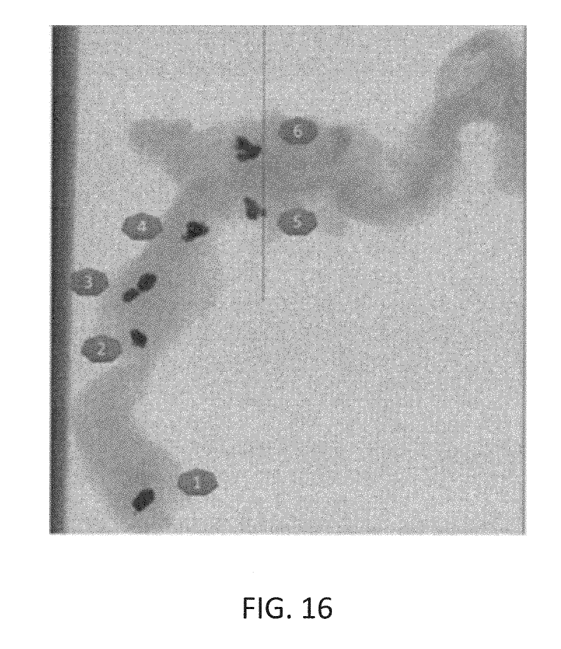

For each of the six dogs, formation of marker clusters was confirmed by X-ray fluoroscopy immediately after injection of the G/CPC paste. Further, the dogs were euthanized 28 days after placement, and the liver, the stomach wall, and the pancreas were extracted from each dog. At the time of injection into the liver, there was no remarkable leakage of the G/CPC paste as observed in Example 12, and cluster formation was observed such that the clusters formed at the injection site were fixed within the liver tissue. The placement in the pancreas also showed a clean formation of clusters of the injected material, which were firmly fixed within the pancreas tissue, strongly suggesting that the G/CPC marker of the present invention is capable of being injected and used inside the pancreas. The marker groups injected inside the stomach wall were each fixed inside the wall and had formed clusters, and as a result of sufficient confirmation by X-ray fluoroscopy, suggested that the G/CPC marker of the present invention is capable of being injected and used inside the gastrointestinal wall. As representative examples, X-ray fluoroscopy images of five G/CPC markers in the liver (FIG. 15), and six G/CPC markers inside the pancreas (FIG. 16) extracted from dogs euthanized 28 days after placement are shown.

In the previous G/CPC markers placed in a dog liver, reflux (leakage) and the like of the G/CPC paste from the injection site occurred, and in many cases, the weight of a large portion of the marker clusters extracted from the liver was about 100 mg, and in particular, the formed clusters were sometimes small and about 50 mg or less. However, here, preparation was performed using Biopex-R Excellent (particle width: 32 μm or less) as the base material of the paste, and therefore formation of clusters of about 200 mg and 300 mg was observed in most cases (FIG. 15) (among clusters (1) to (5) in FIG. 15, clusters of about 200 mg to 300 mg were formed in all except (2)). When a sieved product of Biopex-R Excellent was used, which leads to a G/CPC paste having a high viscosity (10⁸ to 10¹⁰ mPa·s), compared to the case where commercially available Biopex-R was used, reflux from the placement site was reduced, which enabled marker formation proportional to the injection amount. Upon extracting and investigating the G/CPC markers placed inside the pancreas and stomach wall 28 days after placement in a similar manner to the liver, a large portion of the obtained marker clusters were about 150 to 200 mg, and when the variation in the injection amount at the time of placement was also considered, it is thought that the marker clusters existed stably for a period of 28 days.

2-2) Evaluation of Image Recognition Performance of G/CPC Markers Placed in Dog Liver, Inside Stomach Wall, and Pancreas

The G/CPC paste was injected into the dog liver, and image recognition performance was evaluated for the G/CPC markers placed in the liver and extracted after 28 days had elapsed from injection. The extracted liver was placed on an acrylic phantom, and an X-ray fluoroscopy device (X-ray generation device: UD150B-40 manufactured by Shimadzu Corporation; flat panel detector (FPD) for X-ray image acquisition: PaxScan 3030 manufactured by Varian Medical Systems, Inc.) was used to acquire X-ray fluoroscopy images. The 1.5 mm and 2.0 mm diameter pure gold spherical markers (iGold) that are currently used clinically inside the liver and stomach wall were pasted to the extracted liver and stomach as positive controls. The acrylic plate thickness was gradually changed from 1 cm to 25 cm, the tube voltage of the X-ray generation device was fixed at 110 kV, the exposure time was fixed at 3 msec, the tube current was selected from 50 mA, 80 mA and 160 mA depending on the situation, and about 100 X-ray fluoroscopy images were acquired under each of the conditions. A template image was created by cutting out an image of the pure gold particle marker targeted for evaluation from one of the plurality of images from among the X-ray fluoroscopy images obtained under each of the conditions, template pattern matching was performed with respect to the other images by normalized cross-correlation with the template image created beforehand, and when the average value of a correlation coefficient obtained from template pattern matching for about 100 images exceeded 0.3, image recognition was determined to be possible, while image recognition was deemed not possible for lower values. An image processing library (Matrox Imaging Library 9 manufactured by Matrox Corporation) was used for image gradation processing and pattern matching.

(Results)

From a qualitative evaluation by visual inspection of the image, the G/CPC markers were observed to have visibility equivalent to, or better than, the markers used as positive controls. Furthermore, in the evaluation of the image recognition performance of the markers placed inside the liver and the stomach wall by template pattern matching, image recognition performance equivalent to, or better than, the markers used as the positive controls was observed (Table 25 and Table 26). Furthermore, since similar image recognition performance to that of the liver and the stomach wall was obtained for all of the evaluation conditions in the pancreas (Table 27), it is considered that image recognition performance equivalent to, or better than, the markers used as the positive controls can be obtained.

2-3) Safety Evaluation in Dog Having G/CPC Placed Inside Live Liver, Stomach Wall, and Pancreas

Significant bleeding from the liver, the stomach wall, and the pancreas was not observed for a 22 G puncture needle. For the convenience of the operation, the G/CPC paste was injected in a state where the dog abdomen was opened and the liver, the stomach wall, and the pancreas could be directly observed, and therefore blood and biochemical changes were observed that indicated resulting inflammatory reactions such as a transient increase in CRP and total white blood cells, but it may be stated that these were caused by the above operation.

Examples of test value abnormalities reflecting an injury to hepatic parenchymal cells include a transient rise in AST (GOT): aspartate aminotransferase, ALT (GPT): alanine aminotransferase, and ALP: Alkaline phosphatase, but given that the increases are transient and the injection amount is clearly greater than that scheduled to be used clinically (about 30 μL to 100 μL×2 to 3 locations), it may be stated that the placement of the product of the present invention in the liver is toxicologically minor. Furthermore, despite performing placement in the pancreas, there was no specific indication suggesting elevated pancreatic enzymes such as lipase, which can be symptomatic of pancreatitis.

(Summary)

The following objectives, which could not be sufficiently confirmed in previous extracted pig livers and D-PBS(−) circulated extracted pig livers, were able to be confirmed, strongly suggesting that placement is possible as a lesion identification marker for use in radiation therapy in a live body. This test suggested that the marker could be used in not only the liver, but also in the stomach wall and pancreas.

(1) Cluster formation in the G/CPC paste of the present invention occurred rapidly when injected into a dog liver, stomach wall and pancreas, and discharge into the blood stream, and discharge of small clusters into the blood stream, which causes pulmonary obstruction and the like, do not occur.

(2) The obtained G/CPC marker clusters have image recognition performance equivalent to existing gold markers, and further, if an increase in the gold particle content in the paste or an increase in the paste placement amount is achieved, demonstration of an enhanced image recognition performance and tracking performance over existing gold markers can be anticipated even with respect to humans, which have a large torso that is difficult for X-ray fluoroscopy. Of course, X-ray fluoroscopy of easier sites can also be handled by reducing the injection amount.

(3) Even after about 1 month, the formed G/CPC marker clusters are present with good retention of tracking performance.

(4) Based on changes in clinical laboratory values and pathological tissue analysis, transient damage to the liver parenchyma is observed directly after injection, but the damage is minor, and a rapid recovery indicates that the possibility of clinical use is high. The same is true inside the stomach wall and pancreas.

As described above, in the present invention, a lesion identification marker for use in radiation therapy and a lesion identification marker kit for use in radiation therapy could be obtained that enabled pure gold microparticles which absorb X-rays to be placed, with extremely low invasive potential, in any site in the body in an arbitrary amount appropriate for the type of radiation therapy and the therapeutic target site, and that enabled the placement site to be identified over a long period of time by radiation therapy equipment.

TABLE 25
Image recognition performance of G/CPC markers placed in liver

| | | | | | | | iGold | |
|---|---|---|---|---|---|---|---|---|
| | Acrylic plate | | | | | | (spherical) | |
| Condition | thickness/X-ray | | | | | | 1.5 | 2.0 |
| No. | tube current | (1) | (2) | (3) | (4) | (5) | mm | mm |
| 1 | 1 cm/50 mA | ∘ | ∘ | ∘ | ∘ | ∘ | ∘ | ∘ |
| 2 | 5 cm/50 mA | ∘ | ∘ | ∘ | ∘ | ∘ | ∘ | ∘ |
| 3 | 5 cm/80 mA | ∘ | ∘ | ∘ | ∘ | ∘ | ∘ | ∘ |
| 4 | 10 cm/50 mA | ∘ | ∘ | ∘ | ∘ | ∘ | ∘ | ∘ |
| 5 | 10 cm/80 mA | ∘ | ∘ | ∘ | ∘ | ∘ | ∘ | ∘ |
| 6 | 15 cm/50 mA | ∘ | ∘ | ∘ | ∘ | ∘ | ∘ | ∘ |
| 7 | 15 cm/80 mA | ∘ | ∘ | ∘ | ∘ | ∘ | ∘ | ∘ |
| 8 | 20 cm/50 mA | ∘ | x | ∘ | ∘ | x | x | x |
| 9 | 20 cm/80 mA | ∘ | ∘ | ∘ | ∘ | ∘ | x | ∘ |
| 10 | 20 cm/160 mA | ∘ | ∘ | ∘ | ∘ | ∘ | ∘ | ∘ |
| 11 | 25 cm/80 mA | x | x | x | x | x | x | x |
| 12 | 25 cm/160 mA | x | x | x | x | x | x | x |

TABLE 26
Image recognition performance of G/CPC markers placed in stomach

| | | | | | | | | | | | iGold | |
|---|---|---|---|---|---|---|---|---|---|---|---|---|
| | Acrylic plate | | | | | | | | | | (spherical) | |
| Condition | thickness/X-ray | | | | | | | | | | 1.5 | 2.0 |
| No. | tube current | (1) | (2) | (3) | (4) | (5) | (6) | (7) | (8) | (9) | mm | mm |
| 1 | 1 cm/50 mA | ∘ | ∘ | ∘ | ∘ | ∘ | ∘ | ∘ | ∘ | ∘ | ∘ | ∘ |
| 2 | 5 cm/50 mA | ∘ | ∘ | ∘ | ∘ | ∘ | ∘ | ∘ | ∘ | ∘ | ∘ | ∘ |
| 3 | 5 cm/80 mA | ∘ | ∘ | ∘ | ∘ | ∘ | ∘ | ∘ | ∘ | ∘ | ∘ | ∘ |
| 4 | 10 cm/50 mA | ∘ | ∘ | ∘ | ∘ | ∘ | ∘ | ∘ | ∘ | ∘ | ∘ | ∘ |
| 5 | 10 cm/80 mA | ∘ | ∘ | ∘ | ∘ | ∘ | ∘ | ∘ | ∘ | ∘ | ∘ | ∘ |
| 6 | 15 cm/50 mA | ∘ | ∘ | ∘ | ∘ | ∘ | ∘ | ∘ | ∘ | ∘ | ∘ | ∘ |
| 7 | 15 cm/80 mA | ∘ | ∘ | ∘ | ∘ | ∘ | ∘ | ∘ | ∘ | ∘ | ∘ | ∘ |
| 8 | 20 cm/50 mA | x | x | x | x | ∘ | x | x | x | x | x | x |
| 9 | 20 cm/80 mA | x | ∘ | x | x | ∘ | ∘ | x | ∘ | ∘ | x | ∘ |
| 10 | 20 cm/160 mA | ∘ | ∘ | x | ∘ | ∘ | ∘ | ∘ | ∘ | ∘ | ∘ | ∘ |
| 11 | 25 cm/80 mA | x | x | x | x | x | x | x | x | x | x | x |
| 12 | 25 cm/160 mA | x | x | x | x | x | x | x | x | x | x | x |

TABLE 27
Image recognition performance of G/CPC markers
placed in pancreas

| | Acrylic plate | | | | | | |
|---|---|---|---|---|---|---|---|
| Condition | thickness/X-ray | | | | | | |
| No. | tube current | (1) | (2) | (3) | (4) | (5) | (6) |
| 1 | 1 cm/50 mA | ∘ | ∘ | ∘ | ∘ | ∘ | ∘ |
| 2 | 5 cm/50 mA | ∘ | ∘ | ∘ | ∘ | ∘ | ∘ |
| 3 | 5 cm/80 mA | ∘ | ∘ | ∘ | ∘ | ∘ | ∘ |
| 4 | 10 cm/50 mA | ∘ | ∘ | ∘ | ∘ | ∘ | ∘ |
| 5 | 10 cm/80 mA | ∘ | ∘ | ∘ | ∘ | ∘ | ∘ |
| 6 | 15 cm/50 mA | ∘ | ∘ | ∘ | ∘ | ∘ | ∘ |
| 7 | 15 cm/80 mA | ∘ | ∘ | ∘ | ∘ | ∘ | ∘ |
| 8 | 20 cm/50 mA | x | x | x | x | ∘ | x |
| 9 | 20 cm/80 mA | x | ∘ | x | x | ∘ | ∘ |
| 10 | 20 cm/160 mA | ∘ | ∘ | x | ∘ | ∘ | ∘ |
| 11 | 25 cm/80 mA | x | x | x | x | x | x |
| 12 | 25 cm/160 mA | x | x | x | x | x | x |

1. A lesion identification marker for use in radiation therapy comprising a mixture of pure gold particles and a substance containing a calcium phosphate-based bone reinforcing material, or a mixture of pure gold particles, a mixing solution, and a substance containing a calcium phosphate-based bone reinforcing material, wherein
the substance containing a calcium phosphate-based bone reinforcing material has a particle volume mean diameter (MV) of 3 to 12 μm and a D90 of less than 39 μm,
a kneaded material obtained by mixing and kneading a mixing solution and the substance containing a calcium phosphate-based bone reinforcing material in a ratio of 0.4 mL to 1.0 g respectively has a viscosity at 20° C. within a range from 10 to 10¹⁰ mPa·s about 5 min after starting kneading, and
the pure gold particles have an abundance ratio of less than about 3% (volume ratio) for particles having a particle size exceeding about 96 μm.

2. (canceled)

3. The lesion identification marker for use in radiation therapy according to claim 1, wherein
a median diameter (D50) of the pure gold particles is within a range from 16 to 40 μm.

4. The lesion identification marker for use in radiation therapy according to claim 1, wherein
a median diameter (D50) of the pure gold particles is within a range from 20 to 35 μm.

5-13. (canceled)

14. The lesion identification marker for use in radiation therapy according to claim 1, wherein
the mixture is capable of passing through a 20 G to 22 G puncture needle with a needle length of 20 cm.

15. The lesion identification marker for use in radiation therapy according to claim 1, wherein
a mixing ratio by volume of the mixing solution per gram of the substance containing a calcium phosphate-based bone reinforcing material is within a range from about 0.3 mL/g to 0.5 mL/g.

16. The lesion identification marker for use in radiation therapy according to any claim 1, wherein
a weight ratio between the pure gold particles and the substance containing a calcium phosphate-based bone reinforcing material is at least 1:2 but not more than 2:1.

17. (canceled)

18. The lesion identification marker for use in radiation therapy according to claim 1,
comprising at least 5 mg of the pure gold particles.

19-20. (canceled)

21. A lesion identification marker kit for use in radiation therapy comprising pure gold particles and a substance containing a calcium phosphate-based bone reinforcing material, or comprising pure gold particles, a mixing solution, and a substance containing a calcium phosphate-based bone reinforcing material, wherein
the substance containing a calcium phosphate-based bone reinforcing material has a particle volume mean diameter (MV) of 3 to 12 μm and a D90 of less than 39 μm,
a kneaded material obtained by mixing and kneading a mixing solution and the substance containing a calcium phosphate-based bone reinforcing material in a ratio of 0.4 mL to 1.0 g respectively has a viscosity at 20° C. within a range from 10 to 10¹⁰ mPa·s about 5 min after starting kneading, and
the pure gold particles have an abundance ratio of less than about 3% (volume ratio) for particles having a particle size exceeding about 96 μm.

22. (canceled)

23. The lesion identification marker kit for use in radiation therapy according to claim 21, wherein
a median diameter (D50) of the pure gold particles is within a range from 16 to 40 μm.

24. The lesion identification marker kit for use in radiation therapy according to claim 21, wherein
a median diameter (D50) of the pure gold particles is within a range from 20 to 35 μm.

25-33. (canceled)

34. The lesion identification marker kit for use in radiation therapy according to claim 21, wherein
a kneaded material obtained from the pure gold particles and the substance containing a calcium phosphate-based bone reinforcing material, or a kneaded material obtained from the pure gold particles, the mixing solution and the substance containing a calcium phosphate-based bone reinforcing material, is capable of passing through a 20 G to 22 G puncture needle with a needle length of 20 cm.

35. The lesion identification marker kit for use in radiation therapy according to claim 21, wherein
a mixing ratio by volume of the mixing solution per gram of the substance containing a calcium phosphate-based bone reinforcing material is within a range from about 0.3 mL/g to 0.5 mL/g.

36. The lesion identification marker kit for use in radiation therapy according to claim 21, wherein
a weight ratio between the pure gold particles and the substance containing a calcium phosphate-based bone reinforcing material is at least 1:2 but not more than 2:1.

37. (canceled)

38. The lesion identification marker kit for use in radiation therapy according to claim 21,
comprising at least 5 mg of the pure gold particles.

39-40. (canceled)
